SEQUENCE LISTING (2 sequences)

SEQ ID NO: 1 - DNA, 20 bp, synthetic construct
  source 1..20: mol_type = other DNA
cagcagataggctgtccaag

SEQ ID NO: 2 - DNA, 20 bp, synthetic construct
  source 1..20: mol_type = other DNA
gcttcttccagagtctagtc

(19) United States
(12) Patent Application Publication
(10) Pub. No.: US 2024/0374557 A1
(43) Pub. Date: Nov. 14, 2024

(54) BETA ADRENERGIC RECEPTOR COMPOSITIONS AND METHODS OF USE THEREOF

(71) Applicant: THE BOARD OF REGENTS OF THE UNIVERSITY OF TEXAS SYSTEM, Austin, TX (US)

(72) Inventors: Jeffrey M. ZIGMAN, Dallas, TX (US); Joel K. ELMQUIST, Dallas, TX (US); Bharath K. MANI, Dallas, TX (US); Alexandre CARON, Dallas, TX (US)

(73) Assignee: THE BOARD OF REGENTS OF THE UNIVERSITY OF TEXAS SYSTEM, Austin, TX (US)

(21) Appl. No.: 18/686,802

(22) PCT Filed: Aug. 26, 2022
(86) PCT No.: PCT/US2022/075540

§ 371 Date: Feb. 26, 2024

Related U.S. Application Data
(60) Provisional application No. 63/237,683, filed on Aug. 27, 2021.

(51) Int. Cl. A61K 31/357; A61K 31/165; A61K 31/426; A61P 3/06; A61P 3/10

(52) U.S. Cl. CPC A61K 31/357; A61K 31/165; A61K 31/426; A61P 3/06; A61P 3/10

(57) ABSTRACT

Compositions and methods for treatment of one or more metabolic disorders in a subject are provided. Disclosures herein are directed to compositions comprising a beta-adrenergic receptor (ADRB1) antagonist and a beta-3 adrenergic receptor (ADRB3) agonist and methods of using such compositions for treating, preventing, and/or ameliorating a metabolic disease or a metabolic disease condition.

### CROSS REFERENCE TO RELATED APPLICATION

This application claims the benefit of U.S. Provisional Application No. 63/237,683 filed on Aug. 27, 2021, the disclosure of which is hereby incorporated by reference in its entirety.

### ACKNOWLEDGEMENT OF GOVERNMENT SUPPORT

This invention was made with government support under Grant Nos. DK118725, DK103884, DK120894, and DK111644 awarded by the National Institutes of Health. The government has certain rights in this invention.

### STATEMENT REGARDING SEQUENCE LISTING

An electronic version of the Sequence Listing is filed herewith, the contents of which are incorporated by reference in their entirety. The electronic file is 7 kilobytes in size, and titled 106546_735068_SequenceListing.xml.

### BACKGROUND

### 1. Field

The present inventive concept is directed to compositions comprising a beta-1 adrenergic receptor (ADRB1) antagonist and a beta-3 adrenergic receptor (ADRB3) agonist and methods of using such compositions for treating, preventing, and/or ameliorating a metabolic disease or a metabolic disease condition.

### 2. Discussion of Related Art

Obesity and reduced physical activity are major contributors to insulin resistance, diabetes, and diabetes-related complications such as heart disease and renal failure. Control of fatty acid storage and release in adipose tissue is fundamental in energy homeostasis and the development of obesity, type 2 diabetes, and some metabolic diseases. Reduced energy expenditure and resting metabolic rate are predictive of weight gain and metabolic conditions. The sympathetic nervous system (SNS) participates in regulating energy balance in part by controlling adipose tissue function. For example, increased SNS outflow promotes fat mobilization and glucose transport, stimulates non-shivering thermogenesis, promotes browning, and affects the secretion of adipokines. As such, stimulating adipose tissue function by targeting the tissue's adrenergic system could provide for pharmacotherapies to treat obesity, insulin resistance, type 2 diabetes mellitus, and related metabolic diseases. However, such strategies in the field have not been successful as administering ADRB3 agonists in the clinic have had no or only marginal anti-obesity activity. Pursuing adrenergic targets was also deemed less than promising after it was determined that ADRB3 agonists had limited utility in treating obesity due to cardiovascular side effects (e.g., increased blood pressure, elevated heart rate). Further, combination therapies consisting of an ADRB3 agonist with an adrenergic antagonist (e.g., an ADRB1 antagonist) are not considered viable treatment options in the field due to their presumed opposite downstream effects. Accordingly, there remains a need in the field for therapies for treating obesity, insulin resistance, type 2 diabetes mellitus, and similar metabolic related diseases and their complications.

### SUMMARY OF THE INVENTION

The present disclosure is based, in part, on the novel finding that metabolic diseases can be treated by simultaneously activating a beta-3 adrenergic receptor (ADRB3) and antagonizing a beta-1 adrenergic receptor (ADRB1). Accordingly, provided herein are compositions comprising an ADRB3 agonist and an ADRB1 antagonist, methods of using those compositions herein for treating, preventing, or ameliorating a metabolic disease or a metabolic disease condition, and kits used in practicing the methods disclosed herein.

In certain embodiments, the present disclosure provides compositions for treatment of a metabolic disorder in a subject in need thereof. In some embodiments, compositions may comprise at least one beta-1 adrenergic receptor (ADRB1) antagonist and at least one beta-3 adrenergic receptor (ADRB3) agonist. In accordance with these embodiments, an ADRB1 antagonist of compositions disclosed herein may comprise acebutolol, atenolol, betaxolol, bevantolol, bisoprolol, carvedilol, celiprolol, esmolol, labetalol, metoprolol, nadolol, nebivolol, penbutolol, pindolol, propranolol, sotalol, timolol, vortioxetine, or any combination thereof. In some aspects, an ADRB1 antagonist of compositions disclosed herein may comprise any ADRB1 antagonist known to be of clinical use by one of skill in the art. In some embodiments, an ADRB3 agonist of compositions disclosed herein may comprise amibegron, BRL-37344, CL-316243, L-742791, L-796568, LY-368842, mirabegron, Ro40-2148, solabegron, vibegron, ritobegron, KUC-7322 or any combination thereof. In some aspects, an ADRB3 agonist of compositions disclosed herein may comprise any ADRB3 agonist known to be of clinical use by one of skill in the art.

In certain embodiments, the present disclosure provides pharmaceutical compositions for treatment of a metabolic disorder in a subject in need thereof. In some embodiments, pharmaceutical compositions disclosed herein may comprise at least one ADRB1 antagonist, at least one ADRB3 agonist, and at least one pharmaceutical acceptable excipient.

In certain embodiments, the present disclosure provides methods of treating and/or preventing a metabolic disorder in a subject in need thereof. In some embodiments, methods disclosed herein may comprise administering an effective amount of any one of the compositions or pharmaceutical compositions disclosed herein to a subject in need thereof, wherein the subject has or is suspected of having a metabolic disorder. In some embodiments, a subject may have or be suspected of having type 1 diabetes mellitus, type 2 diabetes mellitus, impaired glucose tolerance (IGT), impaired fasting blood glucose (IFG), prediabetes, hyperglycemia, postprandial hyperglycemia, postabsorptive hyperglycemia, overweight, obesity, dyslipidemia, hyperlipidemia, hypercholesterolemia, hypertension, atherosclerosis, endothelial dysfunction, osteoporosis, chronic systemic inflammation, non-alcoholic fatty liver disease (NAFLD), retinopathy, neuropathy, nephropathy, polycystic ovarian syndrome, metabolic syndrome, or any combination thereof.

In some embodiments, methods disclosed herein may reduce blood glucose levels in a subject in need thereof after administering an effective amount of any one of the compositions or pharmaceutical compositions disclosed herein to the subject. In some embodiments, methods disclosed herein may increase plasma insulin levels in a subject in need thereof after administering an effective amount of any one of the compositions or pharmaceutical compositions disclosed herein to the subject as compared to baseline. In some embodiments, methods disclosed herein may reduce body weight in a subject in need thereof after administering an effective amount of any one of the compositions or pharmaceutical compositions disclosed herein to the subject as compared to baseline. In some embodiments, methods disclosed herein may reduce body fat in a subject in need thereof after administering an effective amount of any one of the compositions or pharmaceutical compositions disclosed herein to the subject as compared to baseline. In some embodiments, methods disclosed herein may reduce total cholesterol in a subject in need thereof after administering an effective amount of any one of the compositions or pharmaceutical compositions disclosed herein to the subject as compared to baseline. In some embodiments, methods disclosed herein may reduce LDL cholesterol in a subject in need thereof after administering an effective amount of any one of the compositions or pharmaceutical compositions disclosed herein to the subject as compared to baseline. In some embodiments, methods disclosed herein may reduce triglycerides in a subject in need thereof after administering an effective amount of any one of the compositions or pharmaceutical compositions disclosed herein to the subject as compared to baseline.

In some embodiments, methods disclosed herein may decrease the risk of developing at least one metabolic disorder linked to obesity in a subject in need thereof after administering an effective amount of any one of the compositions or pharmaceutical compositions disclosed herein to the subject. In accordance with such embodiments, methods disclosed herein may decrease the risk of developing type 1 diabetes mellitus, type 2 diabetes mellitus, impaired glucose tolerance (IGT), impaired fasting blood glucose (IFG), prediabetes, hyperglycemia, postprandial hyperglycemia, postabsorptive hyperglycemia, overweight, obesity, dyslipidemia, hyperlipidemia, hypercholesterolemia, hypertension, atherosclerosis, endothelial dysfunction, osteoporosis, chronic systemic inflammation, non-alcoholic fatty liver disease (NAFLD), retinopathy, neuropathy, nephropathy, polycystic ovarian syndrome, metabolic syndrome, or any combination thereof in a subject.

In certain embodiments, the present disclosure provides methods of reducing the dosage of an ADRB3 agonist in a subject in need thereof. In some embodiments, methods disclosed herein may comprise administering to a subject an effective amount of one or more ADRB1 antagonists and one or more ADRB3 agonists, wherein the effective amount of the one or more ADRB3 agonists may be less than the effective amount of the one or more ADRB3 agonist in a subject that is not receiving the one or more ADRB1 antagonists. In some aspects, the effective amount of the one or more ADRB3 agonists may be greater than at least about 1-fold less than the effective amount of the one or more ADRB3 agonists in a subject that is not receiving the one or more ADRB1 antagonists. In some aspects, the effective amount of the one or more ADRB3 agonists may be at least about 2-fold less than the effective amount of the one or more ADRB3 agonists in a subject that is not receiving the one or more ADRB1 antagonists. In some other aspects, the effective amount of the one or more ADRB3 agonists may be at least about 5-fold less than the effective amount of the one or more ADRB3 agonists in a subject that is not receiving the one or more ADRB1 antagonists. In still other aspects, the effective amount of the one or more ADRB3 agonists may be at least about 10-fold less than the effective amount of the one or more ADRB3 agonists in a subject that is not receiving the one or more ADRB1 antagonists.

In certain embodiments, the present disclosure provides methods of reducing untoward effects resulting from beta-3 adrenergic receptor (ADRB3) agonist administration in a subject in need thereof. In some embodiments, methods disclosed herein for reducing at least one untoward effect on the cardiovascular system resulting from ADRB3 agonist administration in a subject in need thereof may comprise administering to the subject an effective amount of one or more ADRB1 antagonists and one or more ADRB3 agonists.

In accordance with these embodiments, methods disclosed herein may comprise administering to the subject an effective amount of one or more ADRB1 antagonists and an effective amount of one or more ADRB3 agonists simultaneously. Further in accordance with these embodiments, methods disclosed herein may comprise administering to the subject an effective amount of one or more ADRB1 antagonists immediately before administering to the subject an effective amount of one or more ADRB3 agonists. In accordance with these embodiments, methods disclosed herein may comprise administering to the subject an effective amount of one or more ADRB1 antagonists immediately after administering to the subject an effective amount of one or more ADRB3 agonists.

In some embodiments, at least one untoward effect on the cardiovascular system resulting from ADRB3 agonist administration in a subject in need thereof may comprise a mean arterial blood pressure (MBP) above normal. In some aspects, a normal MBP can be about 70 mmHg to about 100 mmHg. In some embodiments, at least one untoward effect on the cardiovascular system resulting from ADRB3 agonist administration in a subject in need thereof may comprise a systolic arterial blood pressure (SBP) above normal. In some aspects, a normal SBP can be about 120 mm Hg to about 140 mm Hg. In some embodiments, at least one untoward effect on the cardiovascular system resulting from ADRB3 agonist administration in a subject in need thereof may comprise a diastolic arterial blood pressure (DBP) above normal. In some aspects, a normal DBP can be about 70 mm Hg to about 80 mm Hg.

In some embodiments, methods disclosed herein for reducing at least one untoward effect on the cardiovascular system resulting from ADRB3 agonist administration in a subject in need thereof may comprise administering at least one ADRB3 agonist and at least one ADRB1 antagonist to a subject having or suspected of having a metabolic disorder. In accordance with these embodiments, a subject having or suspected of having a metabolic disorder may have or be suspected of having type 1 diabetes mellitus, type 2 diabetes mellitus, impaired glucose tolerance (IGT), impaired fasting blood glucose (IFG), prediabetes, hyperglycemia, postprandial hyperglycemia, postabsorptive hyperglycemia, overweight, obesity, dyslipidemia, hyperlipidemia, hypercholesterolemia, hypertension, atherosclerosis, endothelial dysfunction, osteoporosis, chronic systemic inflammation, non-alcoholic fatty liver disease (NAFLD), retinopathy, neuropathy, nephropathy, polycystic ovarian syndrome, metabolic syndrome, or any combination thereof.

In some embodiments, methods disclosed herein may improve at least one symptom of a metabolic disorder in a subject while not inducing and/or exacerbating one or more untoward effect on the cardiovascular systems. In some aspects, at least one symptom of a metabolic disorder that may be improved can include a reduction of body fat in the subject, a reduction of fat mass in the subject, a reduction of total cholesterol in the subject, a reduction of LDL cholesterol in the subject, a reduction of triglycerides in the subject, a reduction of glucose in the subject, or any combination thereof.

In certain embodiments, the present disclosure provides methods for maintaining an effective dosage of a beta-3 adrenergic receptor (ADRB3) agonist without inducing at least one untoward effect on the cardiovascular system resulting from ADRB3 agonist administration in a subject in need thereof. In accordance with such embodiments, methods disclosed herein may comprise administering to the subject an effective amount of one or more beta-1 adrenergic receptor (ADRB1) antagonists and one or more ADRB3 agonists, wherein the effective amount of the one or more ADRB3 agonists reduces body fat, reduces total cholesterol, reduces LDL cholesterol, reduces triglycerides, or any combination thereof in the subject in need thereof, and the effective amount of one or more ADRB1 antagonists prevents induction of at least one untoward effect on the cardiovascular system resulting from ADRB3 agonist administration in the subject in need thereof.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein. Embodiments of the present inventive concept are illustrated by way of example in which like reference numerals indicate similar elements.

FIGS. 1A-1N depict representative graphs illustrating that loss of Adrb1 in adipocytes did not exacerbate diet-induced obesity and did not impair adaptation to chronic cold exposure according to various aspects of the disclosure. FIGS. 1A-1H show (FIG. 1A) body weight, (FIG. 1B) lean mass, (FIG. 1C) fat mass, (FIG. 1D) GTT, (FIG. 1E) ITT, weight of (FIG. 1F) eWAT, (FIG. 1G) iWAT and (FIG. 1H) BAT of Ad-Adrb1KO mice and littermate controls after 5 weeks of high fat diet (HDF). FIGS. 1I-1N show (FIG. 1I) body weight, (FIG. 1J) temperature, (FIG. 1K) body composition, weight of (FIG. 1L) eWAT, (FIG. 1M) iWAT and (FIG. 1N) BAT of Ad-Adrb1KO mice and littermate controls after 3 weeks of cold exposure. Results are shown as the mean±SEM for n=5-6 (A-H) and n=7-16 (I-N). *P<0.05, **P<0.01 versus littermate controls assessed using either Student's t test or 2-way ANOVA with Tukey's multiple comparisons test.

FIGS. 2A-2P depict representative graphs illustrating that loss of Adrb1 in adipocytes increased lipid content but did not increase the thermogenic capacity in BAT according to various aspects of the disclosure. FIGS. 2A-2C show (FIG. 2A) body weight, (FIG. 2B) glucose levels, and (FIG. 2C) BAT weight 2 hours after CL-316243 or saline (control) were injected in Ad-Adrb1KO mice or littermate controls. FIGS. 2D-2G show H&E staining of adipocytes from saline-injected littermate control mice (FIG. 2D) or Ad-Adrb1KO mice (FIG. 2E) and CL-316243-injected littermate control mice (FIG. 2F) or Ad-Adrb1KO mice (FIG. 2G) where adipose cells were harvested 2 hours after CL-316243 or saline injection of the mice. FIGS. 2H-2J show Adrb1 mRNA expression in (FIG. 2H) eWAT, (FIG. 2I) iWAT and (FIG. 2J) BAT of Ad-Adrb1KO mice and littermates where tissues were harvested 2 hours after CL-316243 or saline injection of the mice. FIGS. 2K-2M show Ppargc1a mRNA expression in (FIG. 2K) eWAT, (FIG. 2L) iWAT and (FIG. 2M) BAT of Ad-Adrb1KO mice and littermate controls where tissues were harvested 2 hours after CL-316243 or saline injection of the mice. FIGS. 2N-2P show Ucp1 mRNA expression in (FIG. 2N) eWAT, (FIG. 2O) iWAT and (FIG. 2P) BAT of Ad-Adrb1KO mice and littermate controls where tissues were harvested 2 hours after CL-316243 or saline injection of the mice. Results are shown as the mean±SEM for n=4-7. Differences were assessed using 2-way ANOVA.

FIGS. 3A-3E depict representative graphs illustrating that loss of Adrb1 in adipocytes improved the ability of CL-316243 (CL) to lower glycemia according to various aspects of the disclosure. FIGS. 3A-3E show effects of (FIG. 3A) saline, (FIG. 3B) CL 1 mg/kg, (FIG. 3C) CL 0.1 mg/kg, (FIG. 3D) CL 0.01 mg/kg, and (FIG. 3E) CL 0.001 mg/kg administration on blood glucose of Ad-Adrb1KO mice and littermate controls. Results are shown as the mean±SEM for n=6-7. *P<0.05, versus littermate controls assessed using 2-way ANOVA with Tukey's multiple comparisons test.

FIGS. 4A-4J depict representative graphs illustrating that loss of Adrb1 in adipocytes improved the glucose-lowering effects of CL-316243 (CL) independently of changes in NEFA levels according to various aspects of the disclosure. FIGS. 4A-4C show the effects of co-administrating CL with insulin in control littermates and Ad-Adrb1KO mice on blood glucose. FIGS. 4D-4F show the effects of co-administrating CL with glucose in control littermates and Ad-Adrb1KO mice on blood glucose. FIGS. 4G and 4H show insulin levels of littermates and Ad-Adrb1KO mice before and after CL was co-administrated with glucose. FIGS. 4I and 4J show NEFA levels of control littermates and Ad-Adrb1KO mice before and after co-administration of CL with glucose. Results are shown as the mean±SEM for n=5 (FIGS. 4A-4F) and n=5-8 (FIGS. 4G-4J). *P<0.05, ***P<0.0011 versus saline assessed 2-way ANOVA with Tukey's multiple comparisons test.

FIGS. 5A-5F depict representative graphs illustrating that high doses of the ADRB3 agonist, mirabegron, alone or in combination with the ADRB1 antagonist, atenolol, lowered body weight, fat mass, and cholesterol in mice according to various aspects of the disclosure. FIG. 5A shows percent change in body weight in C57BL/6N mice treated as indicated. FIG. 5B shows percent change in fat mass in C57BL/6N mice treated as indicated. FIG. 5C shows amounts of epididymal fat (eWAT) in C57BL/6N mice treated as indicated. FIG. 5D shows total cholesterol levels in C57BL/6N mice treated as indicated. FIG. 5E shows LDL cholesterol levels in C57BL/6N mice treated as indicated. FIG. 5F shows area-under-the-curve blood glucose levels obtained during a glucose tolerance test in C57BL/6N mice treated as indicated.

FIGS. 6A-6C depict representative graphs illustrating that high doses of the ADRB3 agonist, mirabegron, increased blood pressure in mice whereas the same dose of mirabegron in combination with the ADRB1 antagonist, atenolol, did not change blood pressure in mice according to various aspects of the disclosure. FIG. 6A shows systolic blood pressure (SBP) measurements of C57BL/6N mice treated as indicated. FIG. 6B shows diastolic blood pressure (DBP) measurements of C57BL/6N mice treated as indicated. FIG. 6C shows mean arterial blood pressure (MBP) measurements of C57BL/6N mice treated as indicated.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, in part, on the novel finding that metabolic diseases can be treated by pharmacologically targeting the adrenergic system in adipose tissue. In some embodiments, a metabolic disease (e.g., obesity, hypertension, and type 2 diabetes) can be treated by simultaneously activating a beta-3 adrenergic receptor (ADRB3) and antagonizing a beta-1 adrenergic receptor (ADRB1). Accordingly, provided herein are compositions comprising an ADRB3 agonist and an ADRB1 antagonist, methods of using those compositions herein for treating, preventing, and/or ameliorating a metabolic disease or a metabolic disease condition, and kits used in practicing the methods disclosed herein.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, the terms “treat”, “treating”, “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder. As used herein, the term “ameliorate” refers to improve and/or to make better and/or to make more satisfactory (e.g., to improve and/or to make better and/or to make more satisfactory one or more symptoms of a metabolic disease, metabolic disease progression, and/or metabolic disease outcome). As used herein, the phrases “prevention of” and “preventing” refer to avoiding an onset or progression of a disease, disorder, or a symptom thereof.

As used here, the term “activity” is used interchangeably with “functionality” and both terms refer to the physiologic action of a biomolecule. The term “biomolecule” as used herein refers to, but is not limited to, proteins, enzymes, antibodies, DNA, siRNA, and small molecules. “Small molecules” as used herein can refer to chemicals, compounds, drugs, and the like.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

As used herein, the term “agent” refers to a substance (e.g., a biomolecule) that, when administered to a subject, causes one or more chemical reactions and/or one or more physical reactions and/or or one or more physiological reactions and/or one or more immunological reactions in the subject. As used herein, the term “antagonist” refers to an agent (e.g., a biomolecule) that can, directly or indirectly, inhibit a physiologic activity and/or production of a target molecule within a subject, cell, and/or tissue that receives the agent. As used herein, the term “agonist” refers to an agent (e.g., a biomolecule) that can, directly or indirectly, produce a physiologic activity and/or production of a target molecule within a subject, cell, and/or tissue that receives the agent.

As used herein, the term “therapeutically effective amount” refers to the amount of the agent used that is of sufficient quantity to ameliorate, treat and/or inhibit one or more of a disease, disorder or a symptom thereof. The “therapeutically effective amount” will vary depending on the agent(s) used, the route of administration of the agent(s) and the severity of the disease, disorder or symptom thereof. The subject's age, weight and genetic make-up may also influence the amount of the agent that will be a therapeutically effective amount.

It should also be understood that, unless clearly indicated to the contrary, in any methods claimed herein that include more than one step or act, the order of the steps or acts of the method is not necessarily limited to the order in which the steps or acts of the method are recited.

### II. Compositions

The present disclosure provides herein compositions comprising an ADRB3 agonist and an ADRB1 antagonist. In some embodiments, compositions herein may comprise a biomolecule agonist of ADRB3, a biomolecule antagonist of ADRB1, or any combination thereof. In some embodiments, compositions herein may comprise a small molecule agonist of ADRB3, a small molecule antagonist of ADRB1, or any combination thereof. In some embodiments, compositions comprising an ADRB3 agonist and an ADRB1 antagonist may be pharmaceutical compositions.

### (a) Adrenergic Receptors, Adrenergic Antagonists, and Adrenergic Agonists

Adrenergic receptors (adrenoreceptors, ARs) are membrane proteins mediating the actions of epinephrine and norepinephrine. Epinephrine (adrenaline) and norepinephrine (noradrenaline) are neurotransmitters of the sympathetic nervous system and the central nervous system, and function as hormones secreted by the adrenal medulla. There are nine members of adrenergic receptors (α1A, α1B, α1C, α2B, α2C, β1, β2, β3) in humans that are widely distributed throughout the body and play key roles in many important physiological processes. In some embodiments, compositions herein may comprise one or more biomolecules that targets one or more adrenergic receptors. In some embodiments, compositions herein may comprise one or more biomolecules that targets α1A, α1B, α1C, α2B, α2C, β1, β2, β3, or any combination thereof. In some embodiments, compositions herein may comprise one or more biomolecules that targets β1, β2, β3 or any combination thereof. In some embodiments, compositions herein may comprise one or more biomolecules that targets β1, β3, or any combination thereof.

Beta-1 adrenergic receptors (also referred to as “β1,” “β1-AR,” “beta-1-AR,” “Pi adrenoreceptor,” “β1 adrenoceptor” or “ADRB1”) are G-protein coupled receptors (GPCRs) associated with the Gs heterotrimeric G-protein, mediating the catecholamine-induced activation of adenylate cyclase. ADRB1 is membrane-bound and predominantly expressed in cardiac tissue as well as other tissues including, but not limited to adipose tissue. Natural ligands of ADRB1 include epinephrine (adrenaline) and norepinephrine (noradrenaline). In some embodiments, compositions herein may comprise one or more biomolecules that targets ADRB1.

In some embodiments, biomolecules herein that may target ADRB1 can be a peptide, an antibody, a chemical, a compound, a small molecule, an oligo, a nucleic acid molecule, or any combination thereof. Biomolecules that may target ADRB1 can directly inhibit ADRB1 activity, indirectly inhibit ADRB1 activity, inhibit formation of an ADRB1-Gs complex, decrease expression of the ADRB1 gene, decrease expression of the ADRB1 protein, decrease expression of a gene whose transcription is activated—either directly or indirectly—by ADRB1, decrease expression of a protein whose transcription is activated—either directly or indirectly—by ADRB1, decrease protein activation (e.g., protein phosphorylation) either directly or indirectly, by ADRB1, or any combination thereof. In some embodiments, biomolecules herein that may target ADRB1 may act as an antagonist of ADRB1. An antagonist of ADRB1 can directly inhibit ADRB1 activity.

In some embodiments, an antagonist of ADRB1 for use herein can be a small molecule. Non-limiting examples of small molecules that may act as an antagonist of ADRB1 for use herein include acebutolol, atenolol, betaxolol, bevantolol, bisoprolol, carvedilol, celiprolol, esmolol, labetalol, metoprolol, nadolol, nebivolol, penbutolol, pindolol, propranolol, sotalol, timolol, vortioxetine, and the like. In some embodiments, the small molecule may include atenolol, acebutolol, bisoprolol, metoprolol, nadolol, and nebivolol. In some embodiments, an ADRB1 antagonist for use herein can be any ADRB1 antagonist known to be of clinical use by one of skill in the art.

Beta-3 adrenergic receptors (also referred to as “β3,” “β3-AR,” “beta-3-AR,” “β3 adrenoceptor” or “ADRB3”) are G-protein coupled receptors (GPCRs) also associated with the Gs heterotrimeric G-protein, mediating the catecholamine-induced activation of adenylate cyclase. ADRB3 expression has been recorded both at the mRNA and protein levels of several human tissues and its activation involves a variety of cellular pathways. Non-limiting examples of tissues known to express ADRB3 include myocardium, retina, myometrium, adipose tissue, gallbladder, brain, urinary bladder, and blood vessels. In some embodiments, compositions herein may comprise one or more biomolecules that targets ADRB3.

In some embodiments, biomolecules herein that may target ADRB3 can be a peptide, an antibody, a chemical, a compound, a small molecule, an oligo, a nucleic acid molecule, or any combination thereof. Biomolecules that may target ADRB3 can directly increase ADRB3 activity, indirectly increase ADRB3 activity, increase formation of an ADRB3-Gs complex, increase expression of the ADRB3 gene, increase expression of the ADRB3 protein, increase expression of a gene whose transcription is activated—either directly or indirectly—by ADRB3, increase expression of a protein whose transcription is activated—either directly or indirectly—by ADRB3, increase protein activation (e.g., protein phosphorylation) either directly or indirectly, by ADRB3, or a combination thereof. In some embodiments, biomolecules herein that may target ADRB3 may act as an agonist of ADRB3. An agonist of ADRB3 can directly increase ADRB3 activity.

In some embodiments, an agonist of ADRB3 for use herein can be a small molecule. Non-limiting examples of small molecules that may act as an antagonist of ADRB3 for use herein include amibegron, BRL-37344, CL-316243, L-742791, L-796568, LY-368842, mirabegron, Ro40-2148, solabegron, vibegron, ritobegron, KUC-7322, and the like. In some embodiments, the small molecule is mirabegron and/or vibegron. In some embodiments, an ADRB3 agonist for use herein can be any ADRB3 agonist known to be of clinical use by one of skill in the art.

In some embodiments, compositions herein may comprise one or more biomolecules that targets ADRB1, ADRB3, or a combination thereof. In some embodiments, compositions herein may comprise one or more biomolecules that target ADRB1 and one or more biomolecules that target ADRB3. In some embodiments, compositions herein may comprise one or more biomolecules that target ADRB1 and one or more biomolecules that target ADRB3, wherein the biomolecules can be a peptide, an antibody, a chemical, a compound, a small molecule, an oligo, a nucleic acid molecule, or a combination thereof.

In some embodiments, compositions herein may comprise one biomolecule having both ADRB1 antagonist activity and ADRB3 agonist activity.

In some embodiments, compositions herein may comprise one or more biomolecules that target ADRB1 and one or more biomolecules that target ADRB3 wherein the biomolecules may comprise at least two small molecules. In some embodiments, compositions herein may comprise at least one small molecule that targets ADRB1 and at least one small molecule that targets ADRB3 wherein at least one of the small molecules may be an antagonist of ADRB1, an agonist of ADRB3, or a combination thereof. In some embodiments, compositions herein may comprise at least one small molecule that targets ADRB1 and one small molecule that targets ADRB3 wherein at least one of the small molecules is an antagonist of ADRB1 and at least one other one of the small molecules is an agonist of ADRB3.

In some embodiments, compositions herein may have at least one antagonist of ADRB1 comprising acebutolol, atenolol, betaxolol, bevantolol, bisoprolol, carvedilol, celiprolol, esmolol, labetalol, metoprolol, nadolol, nebivolol, penbutolol, pindolol, propranolol, sotalol, timolol, vortioxetine, or any combination thereof and at least one agonist of ADRB3 comprising amibegron, BRL-37344, CL-316243, L-742791, L-796568, LY-368842, mirabegron, Ro40-2148, solabegron, vibegron, ritobegron, KUC-7322, or any combination thereof.

In some embodiments, compositions herein may have at least one antagonist of ADRB1 comprising acebutolol, atenolol, betaxolol, bevantolol, bisoprolol, carvedilol, celiprolol, esmolol, labetalol, metoprolol, nadolol, nebivolol, penbutolol, pindolol, propranolol, sotalol, timolol, vortioxetine, or any combination thereof and at least one agonist of ADRB3 comprising CL-316243. In some embodiments, compositions herein may have at least one antagonist of ADRB1 comprising acebutolol, atenolol, betaxolol, bevantolol, bisoprolol, carvedilol, celiprolol, esmolol, labetalol, metoprolol, nadolol, nebivolol, penbutolol, pindolol, propranolol, sotalol, timolol, vortioxetine, or any combination thereof and at least one agonist of ADRB3 comprising mirabegron. In some embodiments, compositions herein may have at least one antagonist of ADRB1 comprising acebutolol, atenolol, betaxolol, bevantolol, bisoprolol, carvedilol, celiprolol, esmolol, labetalol, metoprolol, nadolol, nebivolol, penbutolol, pindolol, propranolol, sotalol, timolol, vortioxetine, or any combination thereof and at least one agonist of ADRB3 comprising vibegron.

In some embodiments, compositions herein may have at least one antagonist of ADRB1 comprising atenolol and at least one agonist of ADRB3 comprising mirabegron. In some embodiments, compositions herein may have at least one antagonist of ADRB1 comprising metoprolol and at least one agonist of ADRB3 comprising mirabegron. In some embodiments, compositions herein may have at least one antagonist of ADRB1 comprising atenolol and at least one agonist of ADRB3 comprising vibegron. In some embodiments, compositions herein may have at least one antagonist of ADRB1 comprising metoprolol and at least one agonist of ADRB3 comprising vibegron. In some embodiments, compositions herein may have at least one antagonist of ADRB1 comprising atenolol and at least one agonist of ADRB3 comprising CL-316243. In some embodiments, compositions herein may have at least one antagonist of ADRB1 comprising metoprolol and at least one agonist of ADRB3 comprising CL-316243.

In some embodiments, compositions herein may have at least one small molecule antagonist of ADRB1 at about 5 mg to about 500 mg (e.g., about 5 mg, about 10 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg). In some embodiments, compositions herein may have at least one small molecule agonist of ADRB3 at about 5 mg to about 500 mg (e.g., about 5 mg, about 10 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg). In some embodiments, compositions herein may have at least one small molecule antagonist of ADRB1 at about 5 mg to about 500 mg (e.g., about 5 mg, about 10 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg) and at least one small molecule agonist of ADRB3 at about 5 mg to about 500 mg (e.g., about 5 mg, about 10 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg).

### (b) Pharmaceutical Formulations and Treatment Regimens

The compositions disclosed herein for use according to the methods herein described may be provided per se or as part of a pharmaceutical composition, where biomolecules that target ADRB1 and/or ADRB3 can be mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the peptide, and antibody, a chemical, a compound, an oligo, a small molecule, a nucleic acid molecule, or a combination thereof toward targeting ADRB1 and/or ADRB3 accountable for the biological effect. The term “active ingredient” as used herein can also include a genetically modified stem cell as disclosed herein.

### (i) Pharmaceutically Acceptable Carriers and Excipients

Hereinafter, the phrases “physiologically acceptable carrier” and “pharmaceutically acceptable carrier” may be interchangeably used refer to a carrier or a diluent that does not cause significant irritation to an organism and does not abrogate the biological activity and properties of the administered compound. An adjuvant is included under these phrases.

In certain embodiments, compositions disclosed herein may further compromise one or more pharmaceutically acceptable diluent(s), excipient(s), or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art. Pharmaceutical compositions may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, and combinations thereof. Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols. Techniques for formulation and administration of drugs may be found in “Remington's Pharmaceutical Sciences,” Mack Publishing Co., Easton, Pa., latest edition, which is incorporated herein by reference.

In certain embodiments, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In other embodiments, any of the well-known techniques, carriers, and excipients may be used as suitable and as understood in the art.

In certain embodiments, pharmaceutical compositions described herein may be an aqueous suspension comprising one or more polymers as suspending agents. In some aspects, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In other aspects, pharmaceutical compositions disclosed herein may comprise at least about 1%, at least about 5%, at least about 10%, at least about 20%, at least about 25%, at least about 30%, at least about 35%, at least about 40%, at least about 45%, or at least about 50% total amount of polymers as suspending agent(s) by total weight of the composition.

In certain embodiments, pharmaceutical compositions disclosed herein may comprise a viscous formulation. In some aspects, viscosity of the composition may be increased by the addition of one or more gelling or thickening agents. In other aspects, compositions disclosed herein may comprise one or more gelling or thickening agents in an amount to provide a sufficiently viscous formulation to remain on treated tissue. In still other aspects, compositions disclosed herein may comprise at least about 1%, at least about 5%, at least about 10%, at least about 20%, at least about 25%, at least about 30%, at least about 35%, at least about 40%, at least about 45%, or at least about 50% total amount of gelling or thickening agent(s) by total weight of the composition. In yet other aspects, suitable thickening agents can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethylcellulose sodium (CMC), silicon (HPMC), carboxymethyl-cellulose dioxide, polyvinylpyrrolidone (PVP: povidone), Splenda (dextrose, maltodextrin and sucralose), or combinations thereof.

In certain embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least about 1%, at least about 5%, at least about 10%, at least about 20%, at least about 25%, at least about 30%, at least about 35%, at least about 40%, at least about 45%, or at least about 50% total amount of one or more agents by total weight of the composition. In other aspects, one or more of these agents may be added to improve the performance, efficacy, safety, shelf-life and/or other property of the muscarinic antagonist composition of the present disclosure. In some aspects, additives will be biocompatible, and will not be harsh, abrasive, or allergenic.

In certain embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In other aspects, compositions disclosed herein may comprise at least about 1%, at least about 5%, at least about 10%, at least about 20%, at least about 25%, at least about 30%, at least about 35%, at least about 40%, at least about 45%, or at least about 50% total amount of one or more acidifying agents by total weight of the composition.

In certain embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In other aspects, compositions disclosed herein may comprise at least about 1%, at least about 5%, at least about 10%, at least about 20%, at least about 25%, at least about 30%, at least about 35%, at least about 40%, at least about 45%, or at least about 50% total amount of one or more alkalizing agents by total weight of the composition.

In certain embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art. In some aspects, compositions disclosed herein may comprise at least about 1%, at least about 5%, at least about 10%, at least about 20%, at least about 25%, at least about 30%, at least about 35%, at least about 40%, at least about 45%, or at least about 50% total amount of one or more antioxidants by total weight of the composition.

In certain embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents can include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In other aspects, compositions disclosed herein may comprise at least about 1%, at least about 5%, at least about 10%, at least about 20%, at least about 25%, at least about 30%, at least about 35%, at least about 40%, at least about 45%, or at least about 50% total amount of one or more buffering agents by total weight of the composition. In other aspects, the amount of one or more buffering agents may depend on the desired pH level of a composition. In some embodiments, pharmaceutical compositions disclosed herein may have a pH of about 6 to about 9. In other embodiments, pharmaceutical compositions disclosed herein may have a pH greater than about 8, greater than about 7.5, greater than about 7, greater than about 6.5, or greater than about 6.

In certain embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Examples of preservatives can include, but are not limited to, benzalkonium chloride, cetrimide, benzoates (e.g., sodium benzoate), benzyl alcohol, methyl paraben, propyl paraben, alkyl esters, parabens and salts thereof (e.g., methyl or propyl paraben or salts thereof), methyl mercury salts (e.g. borate or nitrate), sodium hypochlorite, parabens, potassium sorbate, sodium benzoate and acetic acid, organic solvents such as ethanol, benzyl alcohol, bronopol, chlorobutanol, propylene glycol, or other preservative systems. In some aspects, any pharmaceutically acceptable preservative can be used. In other aspects, pharmaceutical compositions disclosed herein may comprise at least about 1%, at least about 5%, at least about 10%, at least about 20%, at least about 25%, at least about 30%, at least about 35%, at least about 40%, at least about 45%, or at least about 50% total amount of one or more preservatives by total weight of the composition.

In certain embodiments, pharmaceutical compositions disclosed herein may comprise one or more surface-acting reagents or detergents. In some aspects, surface-acting reagents or detergents may be synthetic, natural, or semi-synthetic. In other aspects, compositions disclosed herein may comprise anionic detergents, cationic detergents, zwitterionic detergents, ampholytic detergents, amphoteric detergents, nonionic detergents having a steroid skeleton, or a combination thereof. In still other aspects, pharmaceutical compositions disclosed herein may comprise at least about 1%, at least about 5%, at least about 10%, at least about 20%, at least about 25%, at least about 30%, at least about 35%, at least about 40%, at least about 45%, or at least about 50% total amount of one or more surface-acting reagents or detergents by total weight of the composition.

In certain embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophanate, zinc oxide, sucrose, glucose, lactose, sorbitol, mannitol, glycerol, polyethylene glycols, sodium caprylate and sodium saccharin and others known to those of ordinary skill in the art. In some aspects, pharmaceutical compositions disclosed herein may comprise at least about 1%, at least about 5%, at least about 10%, at least about 20%, at least about 25%, at least about 30%, at least about 35%, at least about 40%, at least about 45%, or at least about 50% total amount of one or more stabilizers by total weight of the composition.

In certain embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In preferred embodiments, a vapor pressure depression method is used to calculate the osmolarity of the compositions disclosed herein. In some aspects, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In other aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a pharmaceutical composition described herein has an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In still other aspects, pharmaceutical compositions disclosed herein may comprise at least about 1%, at least about 5%, at least about 10%, at least about 20%, at least about 25%, at least about 30%, at least about 35%, at least about 40%, at least about 45%, or at least about 50% total amount of one or more tonicity modifiers by total weight of the composition.

### (ii) Dosage Formulations

Suitable routes of administration may, for example, include oral, rectal, transmucosal, especially transnasal, intestinal or parenteral delivery, including intramuscular, subcutaneous and intramedullary injections as well as, intravenous, intraperitoneal, intranasal injections.

One may administer the pharmaceutical composition in a local or systemic manner, for example, via local injection of the pharmaceutical composition directly into a tissue region of a patient. In some embodiments, a pharmaceutical composition disclosed herein can be administered parenterally, e.g., by intravenous injection, intracerebroventricular injection, intra-cisterna magna injection, intra-parenchymal injection, or a combination thereof. In some embodiments, a pharmaceutical composition disclosed herein can administered to the human patient via at least two administration routes. In some examples, the combination of administration routes by be intracerebroventricular injection and intravenous injection; intrathecal injection and intravenous injection; intra-cisterna magna injection and intravenous injection; and intra-parenchymal injection and intravenous injection.

Pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions. Alternatively, the active ingredient may be in powder form for constitution with a suitable vehicle, e.g., sterile, pyrogen-free water-based solution, before use.

In some embodiments, the pharmaceutical composition or formulation is suitable for oral, buccal or sublingual administration, such as in the form of tablets, capsules, ovules, elixirs, solutions or suspensions, which may contain flavoring or coloring agents, for immediate-, delayed- or controlled-release applications.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavoring agents, coloring matter or dyes, with emulsifying and/or suspending agents and with diluents such as water, ethanol, propylene glycol and glycerin, and combinations thereof.

In some embodiments, the pharmaceutical composition or formulation is suitable for intranasal administration or inhalation, such as delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoro-ethane, a hydrofluoroalkane, carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant. Capsules and cartridges (made, for example, from gelatin) for use in an inhaler or insufflator may be formulated to contain a powder mix of the inhibitor and a suitable powder base such as lactose or starch.

The formulations may be presented in unit-dose or multi-dose containers, for example sealed ampoules or vials, and may be stored in a freeze-dried (lyophilized) condition requiring only the addition of the sterile liquid carrier immediately prior to use.

Pharmaceutical compositions suitable for use in context of the present disclosure may include compositions wherein the active ingredients are contained in an amount effective to achieve the intended purpose. In some embodiments, a therapeutically effective amount means an amount of active ingredients (e.g., ADRB1 antagonists and/or ADRB3 agonists disclosed herein) effective to prevent, slow, alleviate or ameliorate symptoms of a disorder (e.g., a metabolic disorder) or prolong the survival of the subject being treated. Determination of a therapeutically effective amount is well within the capability of those skilled in the art, especially in light of the detailed disclosure provided herein.

For any preparation used in the methods of the present disclosure, the therapeutically effective amount or dose can be estimated initially from in vitro and cell culture assays and or screening platforms disclosed herein. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”).

Dosage amount and interval may be adjusted individually to brain or blood levels of the active ingredient are sufficient to induce or suppress the biological effect (minimal effective concentration, MEC). The MEC will vary for each preparation but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is affected or diminution of the disease state is achieved.

The amount of a composition to be administered will, of course, be dependent on the subject being treated, the severity of the affliction, the manner of administration, the judgment of the prescribing physician, etc. Effective doses may be extrapolated from dose-responsive curves derived from in vitro or in vivo test systems.

### III. Methods of Use

The present disclosure provides for methods of treating, attenuating, and/or preventing a metabolic disorder in a subject in need thereof. In several embodiments, a method for treating, attenuating, or preventing a metabolic disorder in a subject can include administering to a subject, including a human subject, an effective amount of active ingredients (e.g., ADRB1 antagonists and/or ADRB3 agonists disclosed herein).

In certain embodiments, a subject in need thereof can be having, suspected of having, or at risk of having at least one metabolic disorder. As used herein, a “metabolic disorder” refers to a disease and/or condition resulting in the disruption of a subject's metabolic pathway. Non-limiting examples of a metabolic disorder to be treated by the methods herein can include type 1 diabetes mellitus, type 2 diabetes mellitus, impaired glucose tolerance (IGT), impaired fasting blood glucose (IFG), prediabetes, hyperglycemia, postprandial hyperglycemia, postabsorptive hyperglycemia, overweight, obesity, dyslipidemia, hyperlipidemia, hypercholesterolemia, hypertension, atherosclerosis, endothelial dysfunction, osteoporosis, chronic systemic inflammation, non alcoholic fatty liver disease (NAFLD), retinopathy, neuropathy, nephropathy, polycystic ovarian syndrome, metabolic syndrome, or any combination thereof. In some embodiments, a metabolic disorder subject to the methods herein can be obesity, diabetes (e.g., type 2 diabetes mellitus or “T2DM”), pre-diabetes, insulin resistance, hypertension, hypertriglyceridemia, and the like. In some embodiments, a metabolic disorder subject to the methods herein can be a symptom or secondary condition of a metabolic disorder, including but not limited to hypertension, stroke, heart disease, renal failure, fatty liver, hepatic failure, and the like.

A suitable subject includes a human, a livestock animal, a companion animal, a lab animal, or a zoological animal. In some embodiments, the subject may be a rodent, e.g., a mouse, a rat, a guinea pig, etc. In another embodiment, the subject may be a livestock animal. Non-limiting examples of suitable livestock animals may include pigs, cows, horses, goats, sheep, llamas and alpacas. In other embodiments, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In some embodiments, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

In some embodiments, a method for treating, attenuating, or preventing a metabolic disorder in a subject can include administering to a subject an effective amount of at least one ADRB1 antagonist disclosed herein and at least one ADRB3 agonist disclosed herein. In some embodiments, a method for treating, attenuating, or preventing a metabolic disorder in a subject can include administering to a subject a dose of ADRB3 agonist that is effective at reducing body fat, improving glucose tolerance, lowering blood glucose levels, improving dyslipidemia, or the combination thereof but that raises blood pressure and/or heart rate in combination with a dose of ADRB1 antagonist. In some embodiments, a method for treating, attenuating, or preventing a metabolic disorder in a subject can include administering to a subject a dose of ADRB3 agonist that is effective at reducing body fat, improving glucose tolerance, lowering blood glucose levels, improving dyslipidemia, or the combination thereof but that does not raise blood pressure and/or heart rate in combination with a dose of ADRB1 antagonist. The effective amount will depend on the severity of the diseases and the activity of the particular compound(s) employed and is thus within the ordinary skill of the art to determine for any particular host mammal or other host organism. One of skill in the art will appreciate that amounts of ADRB1 antagonists and/or ADRB3 agonists to be administered to a subject in need thereof can vary and be adjusted by one of skill in the art as needed. Suitable amounts for administering an antagonist of ADRB1 according to the methods herein can be at about 0.5 mg to about 1000 mg (e.g., about 0.5 mg, 1 mg, 2 mg, 5 mg, 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg) per day. Suitable amounts for administering an agonist of ADRB3 according to the methods herein can be at about 0.5 mg to about 1000 mg (e.g., about 0.5 mg, 1 mg, 2 mg, 5 mg, 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg) per day.

In some embodiments, methods disclosed herein may administer an antagonist of ADRB1 at about 0.5 mg/day to about 1000 mg/day (e.g., about 0.5 mg/day, about 1 mg/day, about 5 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, about 500 mg/day, about 525 mg/day, about 550 mg/day, about 575 mg/day, about 600 mg/day, about 625 mg/day, about 650 mg/day, about 675 mg/day, about 700 mg/day, about 725 mg/day, about 750 mg/day, about 775 mg/day, about 800 mg/day, about 825 mg/day, about 850 mg/day, about 875 mg/day, about 900 mg/day, about 925 mg/day, about 950 mg/day, about 975 mg/day, about 1000 mg/day).

In some embodiments, methods disclosed herein may administer an agonist of ADRB3 at about 0.5 mg/day to about 1000 mg/day (e.g., about 0.5 mg/day, about 1 mg/day, about 5 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, about 500 mg/day, about 525 mg/day, about 550 mg/day, about 575 mg/day, about 600 mg/day, about 625 mg/day, about 650 mg/day, about 675 mg/day, about 700 mg/day, about 725 mg/day, about 750 mg/day, about 775 mg/day, about 800 mg/day, about 825 mg/day, about 850 mg/day, about 875 mg/day, about 900 mg/day, about 925 mg/day, about 950 mg/day, about 975 mg/day, about 1000 mg/day).

In some embodiments, methods disclosed herein may administer an antagonist of ADRB1 at about 0.5 mg/day to about 1000 mg/day (e.g., about 0.5 mg/day, about 1 mg/day, about 5 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, about 500 mg/day, about 525 mg/day, about 550 mg/day, about 575 mg/day, about 600 mg/day, about 625 mg/day, about 650 mg/day, about 675 mg/day, about 700 mg/day, about 725 mg/day, about 750 mg/day, about 775 mg/day, about 800 mg/day, about 825 mg/day, about 850 mg/day, about 875 mg/day, about 900 mg/day, about 925 mg/day, about 950 mg/day, about 975 mg/day, about 1000 mg/day) and an agonist of ADRB3 at about 0.5 mg/day to about 1000 mg/day (e.g., about 0.5 mg/day, about 1 mg/day, about 5 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, about 500 mg/day, about 525 mg/day, about 550 mg/day, about 575 mg/day, about 600 mg/day, about 625 mg/day, about 650 mg/day, about 675 mg/day, about 700 mg/day, about 725 mg/day, about 750 mg/day, about 775 mg/day, about 800 mg/day, about 825 mg/day, about 850 mg/day, about 875 mg/day, about 900 mg/day, about 925 mg/day, about 950 mg/day, about 975 mg/day, about 1000 mg/day).

In some embodiments, methods disclosed herein may comprise administering to a subject an effective amount of at least one ADRB1 antagonist disclosed herein and at least one ADRB3 agonist disclosed herein wherein the effective amount is sufficient to reduce blood glucose levels in a subject. In some aspects, methods disclosed herein may reduce blood glucose levels in a subject by about 5% or more, by about 10% or more, by about 25% or more, by about 50% or more, by about 75% or more, or by about 100% or more compared to baseline (i.e., the blood glucose levels measured before administration of ADRB1 antagonist and ADRB3 agonist as disclosed herein) in some embodiments. By way of example, an appropriate dose or amount can be determined through use of one or more in vitro or in vivo assays to help identify desirable or optimal dosage ranges or amounts to be administered. For instance, through administration of active ingredients (e.g., ADRB1 antagonists and/or ADRB3 agonists disclosed herein) to a subject, the blood glucose level in the subject can be reduced to a level of about 200 mg/dL or less or about 150 mg/dl or less in some embodiments.

In some embodiments, methods disclosed herein may comprise administering to a subject an effective amount of at least one ADRB1 antagonist disclosed herein and at least one ADRB3 agonist disclosed herein wherein the effective amount is sufficient to increase in plasma insulin levels in a subject. In some aspects, methods disclosed herein may increase in plasma insulin levels in a subject by about 5% or more, by about 10% or more, by about 25% or more, by about 50% or more, by about 75% or more, or by about 100% or more compared to baseline (i.e., the plasma insulin levels measured before administration of ADRB1 antagonist and ADRB3 agonist disclosed herein) in some embodiments.

In some embodiments, methods disclosed herein may comprise administering to a subject an effective amount of at least one ADRB1 antagonist disclosed herein and at least one ADRB3 agonist disclosed herein wherein the effective amount is sufficient to decrease total body weight of a subject. In some aspects, methods disclosed herein may decrease total body weight of a subject by about 1% or more, by about 5% or more, by about 10% or more, by about 25% or more, or by about 50% or more in some embodiments.

In some embodiments, methods disclosed herein may comprise administering to a subject an effective amount of at least one ADRB1 antagonist disclosed herein and at least one ADRB3 agonist disclosed herein wherein the effective amount is sufficient to decrease total body fat of a subject. In some aspects, methods disclosed herein may decrease total body fat of a subject by about 1% or more, by about 5% or more, by about 10% or more, by about 25% or more, or by about 50% or more in some embodiments.

In some embodiments, methods disclosed herein may comprise administering to a subject an effective amount of at least one ADRB1 antagonist disclosed herein and at least one ADRB3 agonist disclosed herein wherein the effective amount is sufficient to decrease total cholesterol in a subject. In some aspects, methods disclosed herein may decrease total cholesterol in a subject by about 1% or more, by about 5% or more, by about 10% or more, by about 25% or more, by about 50% or more, or by about 75% or more compared to baseline (i.e., the total cholesterol measured before administration of ADRB1 antagonist and ADRB3 agonist disclosed herein) in some embodiments. In some embodiments, methods of administration of active ingredients (e.g., ADRB1 antagonists and/or ADRB3 agonists disclosed herein) to a subject as disclosed herein can reduce total cholesterol levels in a subject to under 200 mg/dl or to levels ranging from about 200 mg/dl to about 240 mg/dl (e.g., about 200 mg/dl, about 205 mg/dl, about 210 mg/dl, about 215 mg/dl, about 220 mg/dl, about 225 mg/dl, about 230 mg/dl, about 235 mg/dl, about 240 mg/dl).

In some embodiments, methods disclosed herein may comprise administering to a subject an effective amount of at least one ADRB1 antagonist disclosed herein and at least one ADRB3 agonist disclosed herein wherein the effective amount is sufficient to decrease LDL cholesterol, or low-density lipoprotein cholesterol, in a subject. In some aspects, methods disclosed herein may decrease LDL cholesterol in a subject by about 1% or more, by about 5% or more, by about 10% or more, by about 25% or more, by about 50% or more, or by about 75% or more compared to baseline (i.e., the LDL cholesterol measured before administration of ADRB1 antagonist and ADRB3 agonist disclosed herein) in some embodiments. In some embodiments, methods of administration of active ingredients (e.g., ADRB1 antagonists and/or ADRB3 agonists disclosed herein) to a subject as disclosed herein can reduce LDL cholesterol levels in a subject to under 100 mg/dl or to levels ranging from about 100 mg/dl to about 130 mg/dl (e.g., about 100 mg/dl, about 105 mg/dl, about 110 mg/dl, about 115 mg/dl, about 120 mg/dl, about 125 mg/dl, about 130 mg/dl).

In some embodiments, methods disclosed herein may comprise administering to a subject an effective amount of at least one ADRB1 antagonist disclosed herein and at least one ADRB3 agonist disclosed herein wherein the effective amount is sufficient to decrease triglycerides in a subject. In some aspects, methods disclosed herein may decrease triglycerides in a subject by about 1% or more, by about 5% or more, by about 10% or more, by about 25% or more, by about 50% or more, or by about 75% or more compared to baseline (i.e., the triglycerides measured before administration of ADRB1 antagonist and ADRB3 agonist disclosed herein) in some embodiments. In some embodiments, methods of administration of active ingredients (e.g., ADRB1 antagonists and/or ADRB3 agonists disclosed herein) to a subject as disclosed herein can reduce triglyceride levels in a subject to under 150 mg/dl or to levels ranging from about 150 mg/dl to about 500 mg/dl (e.g., about 150 mg/dl, about 175 mg/dl, about 200 mg/dl, about 225 mg/dl, about 250 mg/dl, about 275 mg/dl, about 300 mg/dl, about 325 mg/dl, about 350 mg/dl, about 375 mg/dl, about 400 mg/dl, about 425 mg/dl, about 450 mg/dl, about 475 mg/dl, about 500 mg/dl).

In some embodiments, methods disclosed herein may comprise administering to a subject an effective amount of at least one ADRB1 antagonist disclosed herein and at least one ADRB3 agonist disclosed herein wherein the effective amount lowers the risk of developing or prevents the development of at least one untoward effect on the cardiovascular system. As used herein, an “untoward effect” refers to a harmful, abnormal, or adverse effect in response to administration of a pharmaceutical composition to a subject. Non-limiting examples of untoward effect on the cardiovascular system includes hypertension, tachycardia, heart failure, left ventricular systolic dysfunction, QT prolongation, myocardial infarction, transient ischemia attack, stroke, and the like. In some embodiments, methods disclosed herein may comprise administering to a subject an effective amount of at least one ADRB1 antagonist disclosed herein and at least one ADRB3 agonist disclosed herein wherein the effective amount lowers the risk of developing hypertension.

In some embodiments, methods disclosed herein may comprise administering to a subject an effective amount of at least one ADRB1 antagonist disclosed herein and at least one ADRB3 agonist disclosed herein wherein the effective amount maintains a normal mean arterial blood pressure (MBP). A normal MBP as used herein refers to a MBP ranging from about 70 mmHg to about 100 mmHg. MBP is calculated from systolic blood pressure (SBP) and diastolic blood pressure (DBP) using formula (I):

$$M B P = 1 / 3 * S B P + 2 / 3 * D B P \qquad ( I )$$

The normal blood pressure for a healthy adult human subject measures between about 120 mmHg and 140 mmHg for SBP and between about 70 mmHg and about 80 mmHg for DBP. Hypertension, or high blood pressure, can be defined as 141 mmHg or higher for SBP and/or or 81 mmHg or higher for DBP.

In some embodiments, methods disclosed herein may comprise administering to a subject an effective amount of at least one ADRB1 antagonist disclosed herein and at least one ADRB3 agonist disclosed herein wherein the effective amount maintains a normal MBP. In some embodiments, methods disclosed herein may comprise administering to a subject an effective amount of at least one ADRB1 antagonist disclosed herein and at least one ADRB3 agonist disclosed herein wherein the effective amount prevents a rise in MBP over baseline (i.e., the MBP measured before administration of ADRB1 antagonist and ADRB3 agonist disclosed herein). In some embodiments, methods disclosed herein may comprise administering to a subject an effective amount of at least one ADRB1 antagonist disclosed herein and at least one ADRB3 agonist disclosed herein wherein the effective amount prevents a clinically significant rise in MBP. As used herein, a clinically significant rise in MBP refers to an increase of MBP greater than 3 mmHg.

In some embodiments, methods disclosed herein may comprise administering to a subject an effective amount of at least one ADRB1 antagonist disclosed herein and at least one ADRB3 agonist disclosed herein wherein the effective amount maintains a normal SBP. In some embodiments, methods disclosed herein may comprise administering to a subject an effective amount of at least one ADRB1 antagonist disclosed herein and at least one ADRB3 agonist disclosed herein wherein the effective amount prevents a rise in SBP over baseline (i.e., the SBP measured before administration of ADRB1 antagonist and ADRB3 agonist disclosed herein). In some embodiments, methods disclosed herein may comprise administering to a subject an effective amount of at least one ADRB1 antagonist disclosed herein and at least one ADRB3 agonist disclosed herein wherein the effective amount prevents a clinically significant rise in SBP. As used herein, a clinically significant rise in SBP refers to an increase of SBP greater than 2 mmHg.

In some embodiments, methods disclosed herein may comprise administering to a subject an effective amount of at least one ADRB1 antagonist disclosed herein and at least one ADRB3 agonist disclosed herein wherein the effective amount maintains a normal DBP. In some embodiments, methods disclosed herein may comprise administering to a subject an effective amount of at least one ADRB1 antagonist disclosed herein and at least one ADRB3 agonist disclosed herein wherein the effective amount prevents a rise in DBP over baseline (i.e., the DBP measured before administration of ADRB1 antagonist and ADRB3 agonist disclosed herein). In some embodiments, methods disclosed herein may comprise administering to a subject an effective amount of at least one ADRB1 antagonist disclosed herein and at least one ADRB3 agonist disclosed herein wherein the effective amount prevents a clinically significant rise in DBP. As used herein, a clinically significant rise in DBP refers to an increase of DBP greater than 1 mmHg.

In certain embodiments, methods of treating, attenuating and/or preventing a metabolic disorder as disclosed herein can be administered immediately before another therapy for a metabolic disorder. Examples of other therapies for metabolic disorders are known by those skill in the art and can include, but are not limited to, diet, exercise, medication, insulin therapy, and the like. In certain embodiments, methods of treating, attenuating and/or preventing a metabolic disorder as disclosed herein can be administered immediately after another therapy for a metabolic disorder. In various embodiments, methods of treating, attenuating and/or preventing a metabolic disorder as disclosed herein can be administered simultaneously as another therapy for a metabolic disorder.

In some embodiments, methods herein may include administration of active ingredients (e.g., ADRB1 antagonists and/or ADRB3 agonists disclosed herein) to a subject who is also receiving insulin therapy. In some aspects, methods of administering active ingredients (e.g., ADRB1 antagonists and/or ADRB3 agonists disclosed herein) to a subject who is also receiving insulin therapy can improve the subject's sensitivity to the insulin therapy.

The present disclosure also provides for methods of reducing the dosage of an ADRB3 agonist to a subject in need thereof by administering an ADRB1 antagonist. It was surprisingly found in the present disclosure that inhibiting ADRB1 can result in a decreased need for ADRB3 agonist. In some embodiments, a method for reducing the dosage of an ADRB3 agonist in a subject can include administering to a subject, including a human subject, an effective amount of one or more ADRB1 antagonists disclosed herein and one or more ADRB3 agonists disclosed herein, wherein the effective amount of the one or more ADRB3 agonist is less than that the effective amount in a subject that is not receiving the one or more ADRB1 antagonists.

In certain embodiments, an effective amount of one or more ADRB3 agonist is about 0.1% to about 99% less than that the effective amount in a subject that is not receiving the one or more ADRB1 antagonists. In some embodiments, the effective amount of the one or more ADRB3 agonist is at least about 0.1%, at least about 0.5%, at least about 1.0%, at least about 1.5%, at least about 2.0%, at least about 2.5%, at least about 3.0%, at least about 4.0%, at least about 4.5%, at least about 5%, at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% less than that the effective amount in a subject that is not receiving the one or more ADRB1 antagonists. In some embodiments, the effective amount of the one or more ADRB3 agonist can be at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, or at least about 100-fold less than that the effective amount in a subject that is not receiving the one or more ADRB1 antagonists.

The present disclosure provides for methods of administering a dosage of an ADRB3 agonist to a subject in need thereof without the need to reduce the dosage due to untoward effects on the cardiovascular system by administering an ADRB1 antagonist. Exemplary data herein showed the unexpected result that inhibiting ADRB1 protected subjects taking high doses of an ADRB3 agonist from developing untoward effects on the cardiovascular system.

In some embodiments, the one or more ADRB3 agonists can be administered concurrently with the one or more ADRB1 antagonists by the same or different modes of administration. In some embodiments, the one or more ADRB3 agonists can be administered before, during, or after the one or more ADRB1 antagonists.

### IV. Kits

The present disclosure provides kits for use in treating or alleviating a target disease, such as a metabolic disorder as described herein. In some embodiments, kits herein can include instructions for use in accordance with any of the methods described herein. The included instructions can comprise a description of administration of a composition containing any of the active ingredients (e.g., ADRB1 antagonists and/or ADRB3 agonists) disclosed herein and optionally the second therapeutic agent, to treat, delay the onset, or alleviate a target disease as those described herein. The kit may further include a description of selecting an individual suitable for treatment based on identifying whether that individual has the target disease (e.g., a metabolic disorder). In still other embodiments, the instructions can include a description of administering an antibody to an individual at risk of the target disease.

The instructions relating to the use of a composition containing active ingredients (e.g., ADRB1 antagonists and/or ADRB3 agonists) disclosed herein can generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as metabolic disorders (e.g., obesity, T2DM). Instructions may be provided for practicing any of the methods described herein. In some embodiments, a kit of the present disclosure may comprise at least one ADRB1 antagonist and at least one ADRB3 agonist in one packaged unit. In some aspects, one packaged unit containing at least one ADRB1 antagonist and at least one ADRB3 agonist may further have instructions for how to administer the least one ADRB1 antagonist and the least one ADRB3 agonist for treating, delaying the onset and/or alleviating a metabolic disorder (e.g., obesity, T2DM). In accordance with these embodiments, instructions included in the one packaged unit may advise the subject to be treated and/or the professional caretaker (e.g., physician, nurse, pharmacist, and the like) of the subject to be treated on how to administer the least one ADRB1 antagonist and the least one ADRB3 agonist in a manner effective for treating, delaying the onset and/or alleviating a metabolic disorder (e.g., obesity, T2DM) in the subject. In some embodiments, instructions included in the one packaged unit may advise the subject to be treated and/or the professional caretaker (e.g., physician, nurse, pharmacist, and the like) of the subject to be treated to administer the least one ADRB1 antagonist and the least one ADRB3 agonist simultaneously, to administer the least one ADRB1 antagonist before the least one ADRB3 agonist, or to administer the least one ADRB1 antagonist after the least one ADRB3 agonist. As an example, one packaged unit disclosed herein may contain: 1) a ADRB1 antagonist in tablet form and a ADRB3 agonist in tablet form wherein each tablet is in a blister pack; and 2) instructions as to dosing schedule for administration of the tablets to the subject in need thereof for the intended treatment (e.g., a metabolic disorder).

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). In some examples, at least one active agent in the composition can be an ADRB1 antagonist and/or an ADRB3 agonist as described herein.

Kits may optionally provide additional components such as buffers and interpretive information. Normally, the kit includes a container and a label or package insert(s) on or associated with the container. In some embodiments, the invention provides articles of manufacture comprising contents of the kits described above.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

Introduction to Examples 1-3: The sympathetic nervous system (SNS) plays a critical role in controlling adipose tissue function. Increased SNS outflow promotes fat mobilization and glucose transport, stimulates non-shivering thermogenesis, promotes browning, and affects the secretion of adipokines. Many of these effects are attributed to norepinephrine activation of the adrenergic receptor 33 (ADRB3). However, global deletion of Adrb3 in mice does not affect basal metabolic rate or adaptive response to cold and diet-induced obesity (DIO), suggesting that another adrenergic receptor may be important. One such candidate is the adrenergic receptor β1 (ADRB1). However, the physiological relevance of the potential crosstalk between ADRB1 and ADRB3 in adipocytes is still unsettled.

### Example 1

The direct role of adipose ADRB1 in the metabolic adaptation to DIO and cold exposure was addressed using a transgenic mouse model. Specifically, a transgenic mouse model having a selective deletion of Adrb1 in adipocytes was generated to determine if Adrb1 in adipocytes could exacerbate DIO in mice. In brief, a C57BL/6J congenic version of the adiponectin (Adipoq)-Cre mouse1 was crossed with Adrb1flox/flox mice2 to generate Ad-Adrb1KO mice.

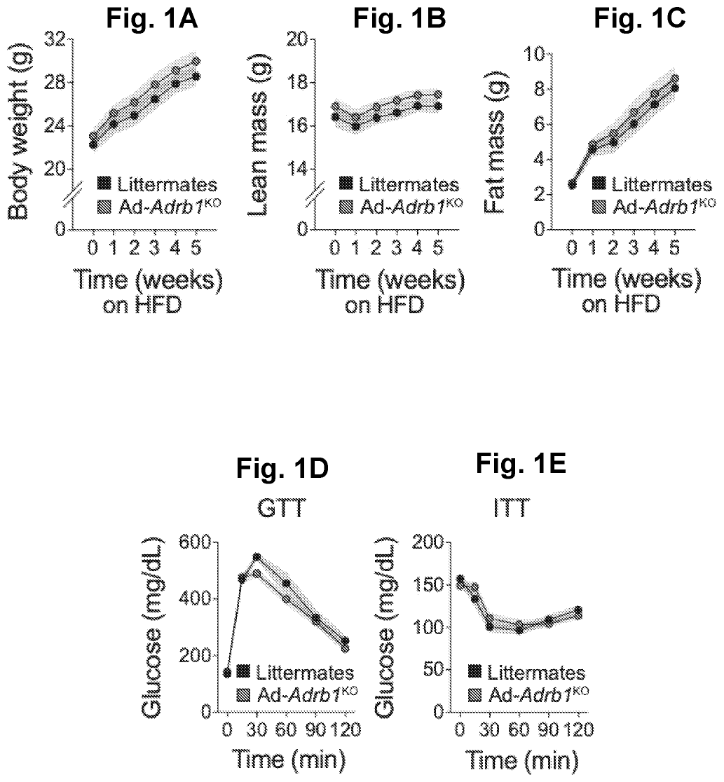

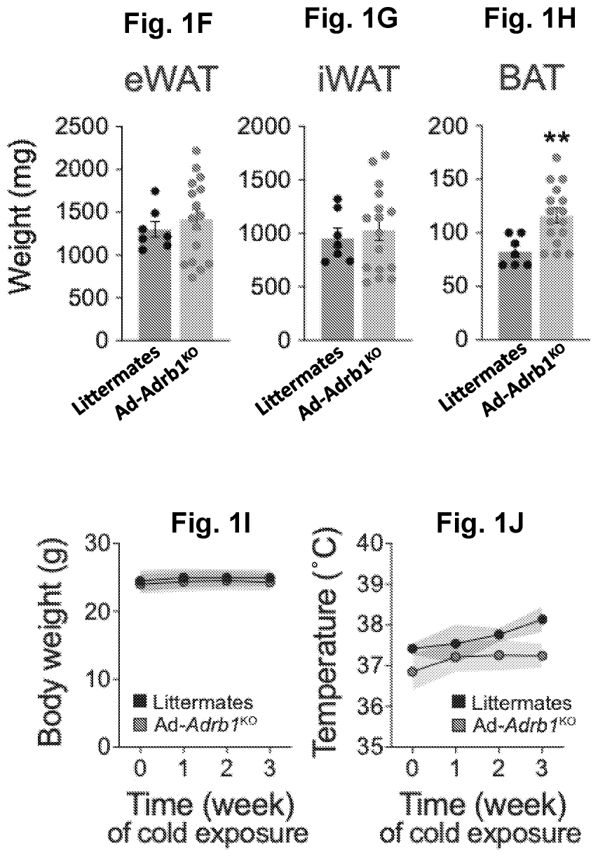

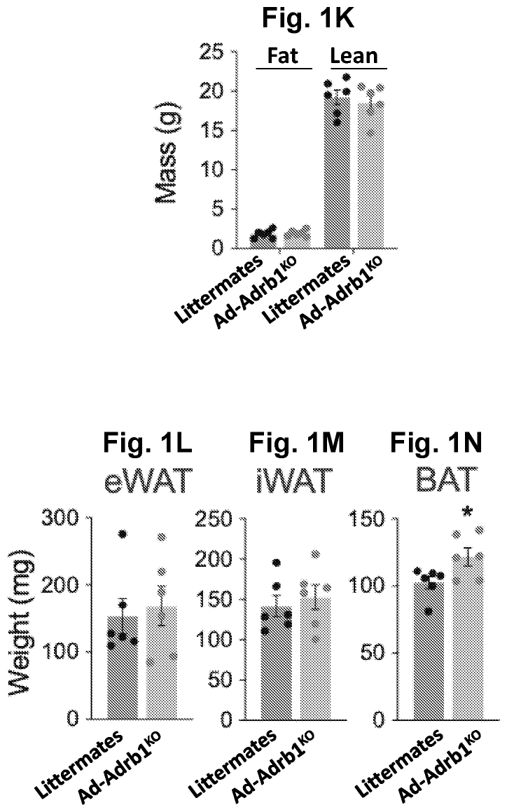

Deletion of Adrb1 in adipose tissue did not affect the susceptibility to DIO in Ad-Adrb1KO mice fed a high fat diet (HFD) for 5 weeks (FIG. 1A). Body composition, glucose tolerance and insulin sensitivity were similar between genotypes after the obesogenic regimen (FIGS. 1B-1E). Similarly, the weight of the epididymal (eWAT) and inguinal (iWAT) white adipose tissue were unchanged (FIGS. 1F-1G). However, Ad-Adrb1KO mice exhibited enlarged interscapular BAT (FIG. 1H). These data indicated that the absence of Adrb1 in adipose tissue did not exacerbate DIO but resulted in increased BAT mass.

BAT is a key regulator of non-shivering thermogenesis.3,4 Altered BAT activity results in cold intolerance and deficit in energy expenditure.3,4 and mice with a global Adrb1 deletion exhibit hypothermia and impaired BAT functioning.5 As such, it was assessed if the observed increase in BAT mass in Ad-Adrb1KO mice (FIG. 1H) could affect adaptive thermogenesis in response to cold exposure.

As shown in FIGS. 11 and 1J, Ad-Adrb1KO mice exposed to a thermal environment of 6° C. for 3 weeks did not exhibit changes in body weight nor body temperature. Likewise, body composition was unaltered (FIG. 1K). Similarly, eWAT (FIG. 1L) and iWAT (FIG. 1M) masses were unchanged, but BAT weight remained significantly larger in Ad-Adrb1KO mice (FIG. 1N). Altogether, these data suggested that the loss of adipose tissue Adrb1 did not affect cold tolerance but resulted in increased BAT mass even in cold-acclimated animals. These results were quite unexpected considering that mice treated with an ADRB1 antagonist were reported to be unable to defend body temperature when exposed to a cold challenge.6,7 What these differences showed was that Adrb1 in non-adiponectin-expressing cells was important for adaptation to diet and cold exposure.

### Example 2

Physiological relevance of crosstalk between ADRB1 and ADRB3 in adipocytes was assessed. In brief, Ad-Adrb1KO mice and littermate controls were injected with either saline (control) or an agonist of ADRB3 (CL-316243-0.1 mg/kg). CL-316243 (5-[(2R)-2-[[(2R)-2-(3-Chlorophenyl)-2-hydroxyethyl]amino]propyl]-1,3-benzodioxole-2,2-dicarboxylic acid disodium salt) is a potent and highly selective ADRB3 agonist (EC50=3 nM) with over 10000-fold selectivity for ADRB3 over ADRB1 and ADRB2. Four hours after CL-316243 injection, mice were sacrificed and BAT depots were dissected.

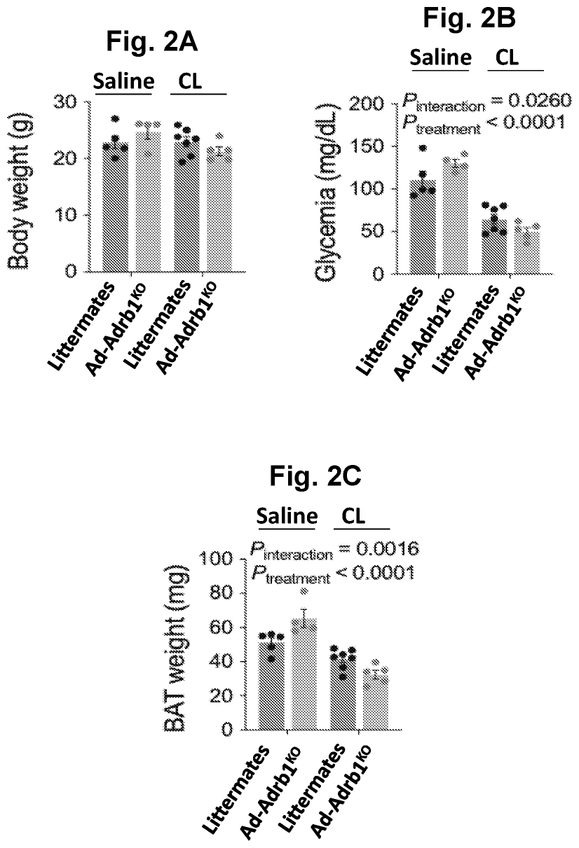

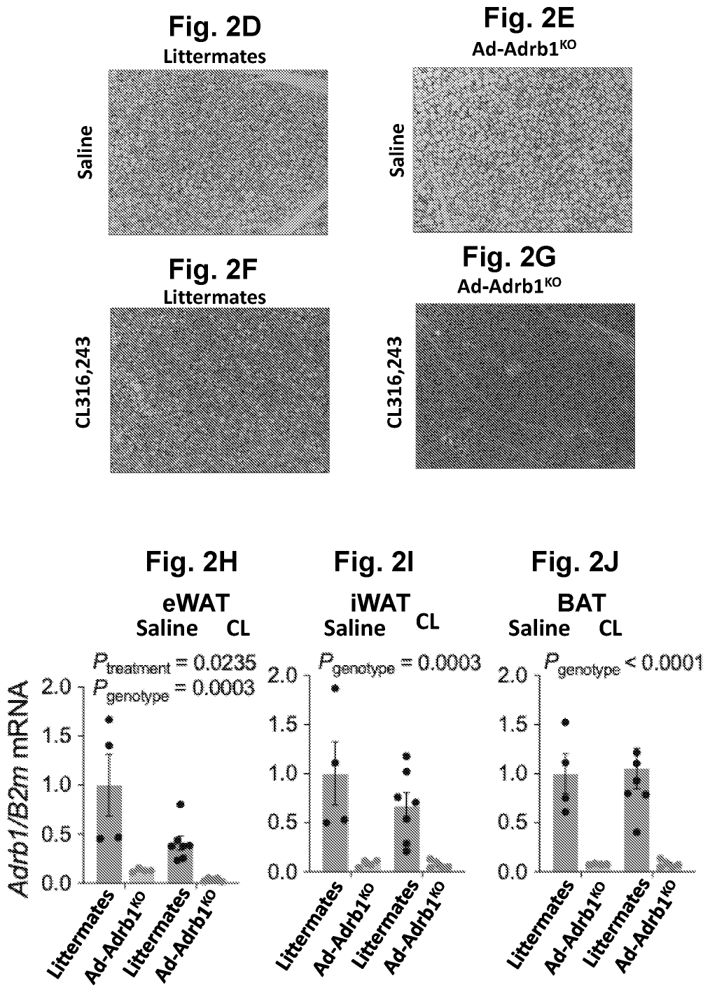

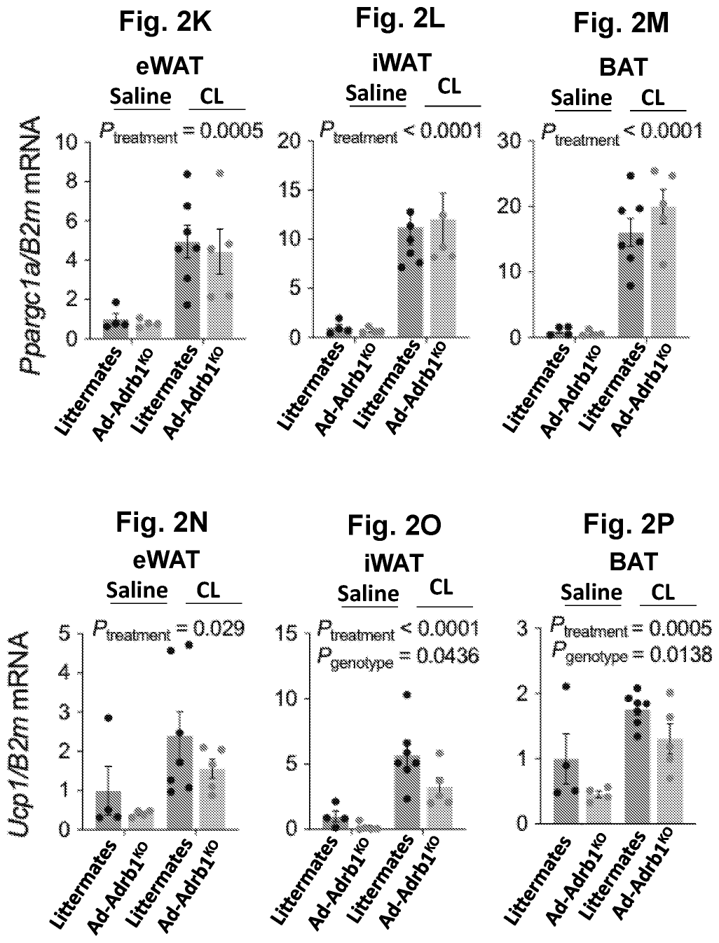

As shown in FIG. 2A, this acute treatment with CL-316243 did not significantly change the weight of the mice. However, there was a significant interaction for the efficiency of CL-316243 to reduce blood glucose levels (FIG. 2B). Saline-treated Ad-Adrb1KO mice had enlarged BAT mass whereas BAT mass in CL-316243-treated Ad-Adrb1KO mice was comparable to CL-316243-treated littermates (FIG. 2C). Histological evaluation of BAT following H&E (hematoxylin and eosin) staining (FIGS. 2D-2G) indicated that the enlargement in BAT was the result of an apparent increase in lipid content within the BAT (FIGS. 2D and 2E). Interestingly, significant interaction between the treatment and the genotype (P=0.0016) was observed, suggesting that although Ad-Adrb1KO mice had a larger BAT at baseline, CL-316243-induced mobilization of intracellular triglycerides was markedly improved (FIG. 2C). Supporting this observation, a near-complete depletion of lipid droplets was observed in the H&E-stained BAT after CL-316243 treatment in mice lacking Adrb1 in adipocytes (FIG. 2G). These data showed that the ability of CL-316243 to reduce BAT lipid content was improved in the absence of Adrb1 and supported a model of significant crosstalk between ADRB1 and ADRB3 in regulating lipolysis.

Next, the possibility that acute CL-316243 treatment impacted Adrb1 expression in different adipose depots was evaluated. CL-316243 treatment significantly reduced Adrb1 mRNA expression in eWAT (FIG. 2H), but not in iWAT (FIG. 2I) or BAT (FIG. 2J) of littermate controls. Adrb1 mRNA expression was absent in adipose depots of Ad-Adrb1KO mice (FIGS. 2H-2J). An increase in peroxisome proliferator-activated receptor gamma coactivator a-alpha (Ppargc1a) mRNA expression (FIGS. 2K-2M) and uncoupling protein 1 (Ucp1) mRNA expression (FIGS. 2N-2P) was observed in every adipose depot following CL-316243 treatment, independent of the genotype. Surprisingly, the mRNA expression of Ucp1 was significantly lower in iWAT (FIG. 2O) and BAT (FIG. 2P) of Ad-Adrb1KO mice, suggesting a reduction in the thermogenic capacity of these depots in the absence of ADRB1. Collectively, these data suggested that the loss of Adrb1 may overall reduce the thermogenic capacity and does not promote BAT thermogenic capacity in response to an ADRB3 agonist.

### Example 3

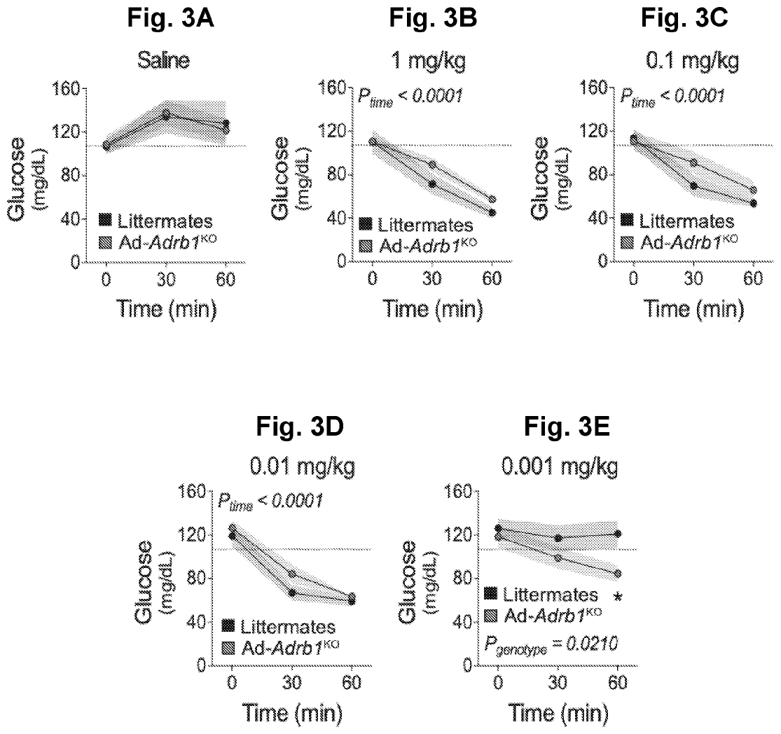

Crosstalk between ADRB3 and ADRB1 in adipose tissue for the control of insulin release and glucose clearance was assessed. To determine if the absence of functional ADRB1 in BAT sensitizes mice to the capacity of an ADRB3 agonist to lower blood glucose, Ad-Adrb1KO mice and littermate controls were first administered either saline (control) (FIG. 3A), 1 mg/kg CL-316243 (FIG. 3B), 0.1 mg/kg CL-316243 (FIG. 3C), 0.01 mg/kg CL-316243 (FIG. 3D), or 0.001 mg/kg CL 316243 (FIG. 3E), and glucose levels were monitored over the 60-minute period following administration. CL-316243 had a glucose lowering effect observed in both the genotypes of mice even at a low dose of 0.01 mg/kg. Interestingly, a very low dose of CL-316243 (0.001 mg/kg) was still effective in reducing glycemia only in Ad-Adrb1KO mice, but not littermate controls.

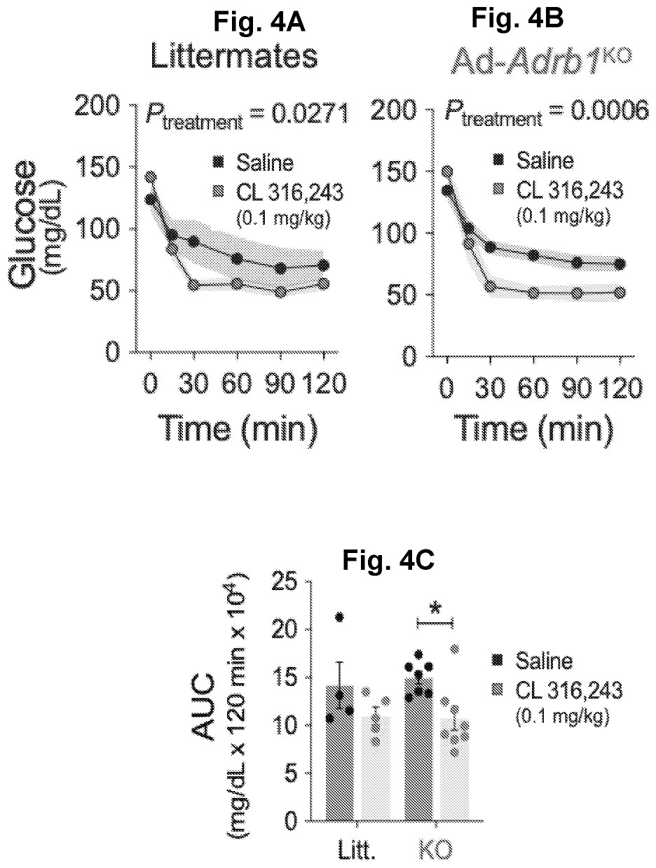

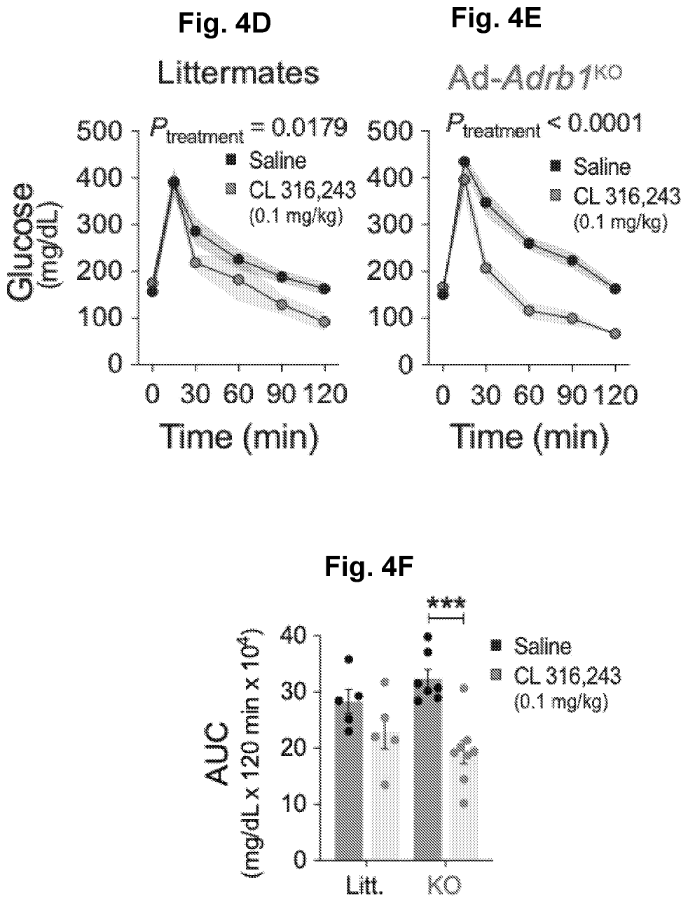

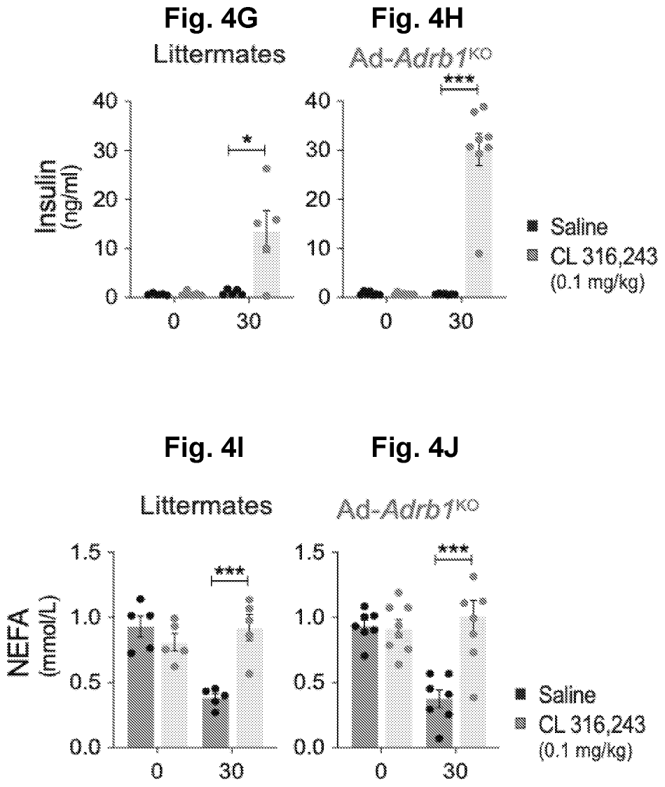

Using an intermediate dose of CL-316243 (0.1 mg/kg) in combination with insulin, a slight but significant improvement in insulin sensitivity was observed in Ad-Adrb1KO mice compared to control littermates (FIGS. 4A-4C). Glucose clearance was also drastically improved in Ad-Adrb1KO mice when the intermediate dose of CL-316243 (0.1 mg/kg) was co-administrated with glucose (FIGS. 4D-4F). Co-administration of CL-316243 (0.1 mg/kg) and glucose also led to a striking increase in plasma insulin levels (43-fold in Ad-Adrb1KO mice versus 12-fold in littermate controls, FIGS. 4G-4H).

CL-316243 effects on incretin were previously shown to be dependent on non-esterified fatty acids (NEFA) release.8,9 However, co-administrating CL-316243 with glucose had no difference on plasma NEFA levels between Ad-Adrb1KO mice and littermate controls (FIGS. 4I-4J). Together, these data demonstrated a previously unrecognized crosstalk between ADRB3 and ADRB1 in adipose tissue for the control of insulin release and glucose clearance, which was independent of NEFA release.

Discussion of Examples 1-3: Exemplary methods provided in Examples 1-3 herein established a novel crosstalk between ADRB1 and ADRB3 in adipose tissue for the control of glucose homeostasis. Identification of this crosstalk between ADRB1 and ADRB3 in adipose tissue provide support for administration of a combined ADRB1 antagonist with an ADRB3 agonist for the treatment of metabolic disorders.

### Methods Used in Examples 1-3

Animal care. Mice were housed at an ambient temperature of 23±1° C. and maintained on a 12 hour light/dark cycle (lights on 0600-1800) and fed with normal mouse chow diet (Envigo, 2016 Teklad global 16% protein rodent diets) unless otherwise stated. All experiments were conducted using 8-14-week-old males.

Generation of Ad-Adrb1KO mice. To generate mice in which Adrb1 selectively deleted from adipose tissue (Ad-Adrb1KO), a C57BL/6J congenic version of the adiponectin (Adipoq)-Cre mouse (The Jackson Laboratory, Stock No: 028020) was crossed with Adrb1flox mice. The following genotyping primers were used: forward, 5′-CAG CAG ATA GGC TGT CCA AG-3′ (SEQ ID NO: 1) and reverse, 5′-GCT TCT TCC AGA GTC TAG TCT-3′ (SEQ ID NO: 2).

Diet-induced obesity model. Mice were fed a high-fat diet (HFD, 60% kCal from fat, Research Diets, D12492) for 5 weeks starting at 8-10 weeks-old. Body composition was assessed every 13 weeks by nuclear magnetic resonance (NMR) spectroscopy (Bruker Minispec mq10 NMR 0.23 T/10 MHz). After 5 weeks on HFD, a glucose tolerance test (GTT) and an insulin tolerance test (ITT) were performed as described below.

Glucose homeostasis. For the GTT, mice were fasted for 6 hours and were injected (intraperitoneal) with 1.5 g/kg of d-Glucose (Sigma 49163-100ML). For the ITT, animals were fasted for 6 hours and were injected with 0.75 mU/kg (intraperitoneal) of human recombinant insulin (Eli Lilly). Tail vein blood glucose levels were then monitored using a glucometer (Bayer's Contour Blood Glucose Monitoring System.

Chronic cold exposure. Singly housed 9-12 week-old mice fed chow were placed in cold chambers maintained at 6° C. for 3 weeks to study the metabolic adaptation of Ad-Adrb1KO mice to chronic cold exposure. Five days before placing the mice in cold chambers, the mice were implanted subcutaneous (s.c.) with IPTT-300 temperature transponders (Bio Med Data Systems, Seaford, DE) on the back for body temperature measurements. Food intake, body weight, body temperature measurements were made weekly. Body temperature measurements were made using a hand-held reader (Bio Med Data Systems) for the transponder. After measurement of body composition using NMR spectroscopy (Bruker Minispec mq10 NMR 0.23 T/10 MHz) at the end of the 3-week study, the mice were sacrificed.

Treatment with CL 316, 243. Mice were fasted for 2 hours (0800-1000) and then given an injection of the ADRB3 agonist CL 316,243 (“CL”, 0.001-1 mg/kg) or vehicle (saline). In some cohorts, human recombinant insulin (Eli Lilly) or D-glucose (Sigma 49163-100ML) was co-administrated with CL.

Histology. Samples from BAT were taken 4 hours following CL (0.1 mg/kg) or vehicle (saline) and were immediately fixed overnight in 10% formalin. Samples were then dehydrated, embedded in paraffin, and cut into 5 pm-thick sections. Sections were stained with hematoxylin and eosin (H&E) to perform general histology. All pictures were taken at a magnification of 20× on a Zeiss Axioskop 2 microscope connected to a digital camera (AxioCam; Zeiss, Thornwood, NY). Only the sharpness, contrast, and brightness were adjusted.

Quantitative real-time PCR. Ad-Adrb1KO mice and littermate controls were injected with CL 316,243 (0.1 mg/kg) after a 2 hour fast, and tissues were collected 4 hours later. Total mRNA was isolated from eWAT, iWAT and interscapular BAT using the Aurum™ Total RNA Fatty and Fibrous Tissue Kit (Bio-Rad, 732-6830). RNA concentration was estimated from absorbance at 260 nm. cDNA synthesis was performed using the iScript Advanced cDNA Synthesis Kit (Bio-Rad, 172-5038). mRNA extraction and cDNA synthesis were performed following the manufacturer's instructions. cDNA was diluted in DNase-free water before quantification by real-time PCR. Relative quantification of gene expression was performed on diluted cDNA in duplicate samples using a CFX384 Touch™ real-time PCR (Bio-Rad). Fold differences in targeted mRNA expression were calculated using the 2-delta cycle threshold method and data were normalized to beta-microglobulin (B2m) expression. TaqMan gene expression assays for Adrb1 (Mm00431701_s1), B2m (Mm00437762_m1), Ppargc1a (Mm01208835_m1), and Ucp1 (Mm01244861_m1) were purchased from ThermoFisher Scientific.

Evaluation of insulin and NEFA levels. Blood from tail vein was collected before and 30 minutes after co-administration of CL with D-glucose into EDTA tubes. Plasma was isolated by centrifugation (4000 g×10 minutes at 4° C.). Plasma insulin (Mouse Ultrasensitive Insulin ELISA, ALPCO, 80-INSMSU-E01) and NEFA (FUJIFILM Wako Diagnostics-NEFA Reagent, 999-34691, 995-34791, 991-34891, 993-35191) levels were measured following manufacturer recommendations.

Statistics. Data are expressed as the mean±SEM. Comparisons between two experimental conditions were analyzed by Student's unpaired t test. Two-way ANOVA followed by Tukey post-hoc test was used to compare more than two experimental conditions. All statistical tests were performed using GraphPad Prism (version 7.0), and p<0.05 was considered statistically significant.

### REFERENCES USED IN EXAMPLES 1-3

1. Eguchi et al., Cell Metab. 2011; 13(3): 249-59.
2. Mani et al., J Clin Invest. 2016; 126(9): 3467-78.
3. Cannon and Nedergaard, Physiol Rev. 2004; 84(1): 277-359.
4. Collins and Surwit, Recent Prog Horm Res. 2001; 56:309-28.
5. Ueta et al., J Endocrinol. 2012; 214(3): 359-65.
6. Soloveva et al., Mol Endocrinol. 1997; 11(1): 27-38.
7. Jiang et al., eLife. 2017; 6.
8. MacPherson et al., Am J Physiol—Endoc M. 2014; 307(7):E563-70.
9. Heine et al., Cell Metab. 2018; 28(4): 644-55 e4.

### Example 4

Untoward effects on the cardiovascular system can limit use of high doses of beta-3 receptor agonists. For example, doses of mirabegron—an ADRB3 agonist—higher than those currently approved for overactive bladder (25 mg-50 mg) are not commonly administered to patients due to the risk of undesired effects impacting the cardiovascular system, such as high blood pressure and elevated heart rate. Thus, despite the promise that ADRB3 agonists have for the treatment of metabolic disorders, one of skill in the art would avoid administering ADRB3 agonists at concentrations that could, for example, effectively lower blood glucose for fear of elevating blood pressure to dangerous levels.

A prescribing physician may look to co-administer one or more drugs with an ADRB3 agonist to control the rising blood pressure; however, no one of skill in the art would look to co-administer an ADRB3 agonist with an ADRB1 antagonist as it is commonly understood that these two drugs would trigger opposite, conflicting downstream effects.

Exemplary data provided in Examples 1-3 herein were the first to identify a novel crosstalk between ADRB1 and ADRB3 in adipose tissue. In view of this new discovery, the exemplary data of Example 4 examined the physiological effects of co-administering high doses of an ADRB3 agonist and an ADRB1 antagonist contrary to the current dogma in the field.

In brief, three cohorts of male C57BL/6N mice, including a total of 100 mice, were used in these studies. Five-week-old mice were placed on a 42% high fat diet for 4 weeks. The mice were then randomized to one of 4 treatment groups as shown in Table 1 provided below. The treatments were provided for 4 weeks, during which time mice were maintained on the 42% high fat diet.

TABLE 1

| Treatment Group | Drug 1 | Drug 2 |
|---|---|---|
| 1 | Placebo | Placebo |
| | (5% PEG 400) | (water) |
| 2 | Mirabegron | Placebo |
| | 8 mg/kg | (water) |
| 3 | Placebo | Atenolol |
| | (5% PEG 400) | 60 mg/kg |
| 4 | Mirabegron | Atenolol |
| | 8 mg/kg | 60 mg/kg |

Mirabegron, an ADRB3 agonist, was administered at an 8 mg/kg daily dose. Atenolol is an ADRB1 antagonist and was administered to the mice at, on average, 60 mg/kg per day. Mirabegron or its buffer (5% PEG 400; placebo) were administered to the mice daily by gavage for 4 weeks. Mice were also given ad libitum access to a water bottle or a water bottle containing atenolol. Those mice with access to water bottles with atenolol self-administered on average 60 mg/kg per day. After the treatment period, body weight, food intake, body composition, oral glucose tolerance testing results, total cholesterol, and blood pressure measurements were collected for each mouse in the study. Oral glucose tolerance testing was performed on overnight fasted mice administered 5 μL 60% glucose solution per g BW using a gavage tube; blood glucose levels were determined with a glucometer using blood from tail nicks at times 0, 15, 30, 60 and 120 minutes. Blood pressure measurements were obtained using a CODA-HT8 High Throughput Noninvasive Blood Pressure system with 8 activated channels for use with mice and rats from Kent Scientific. Methods and statistical analysis were performed as described above.

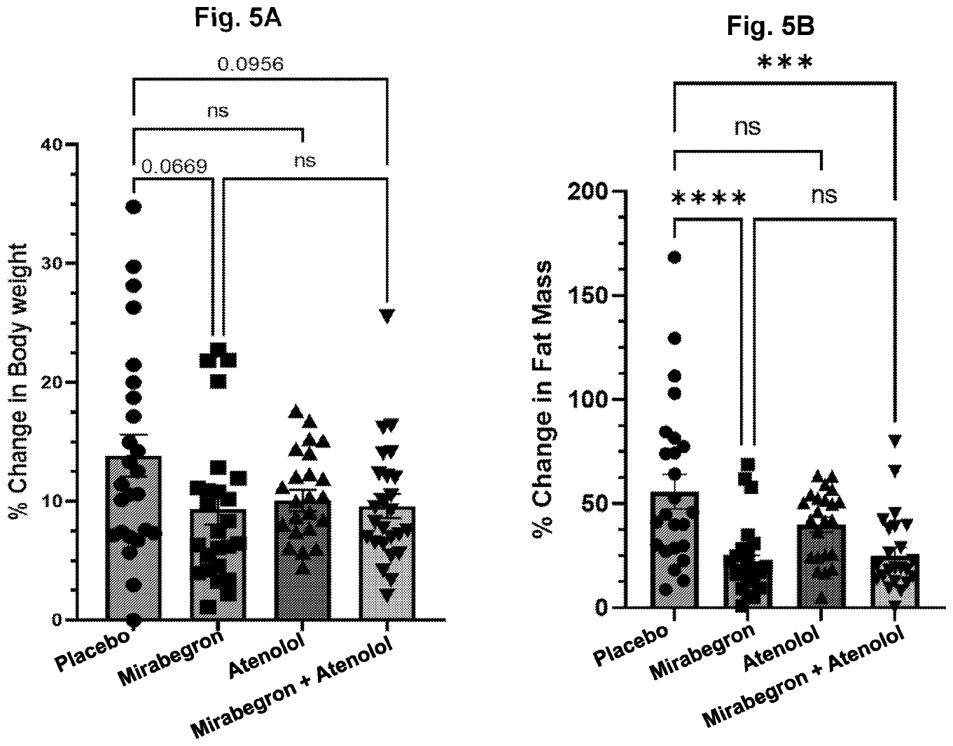

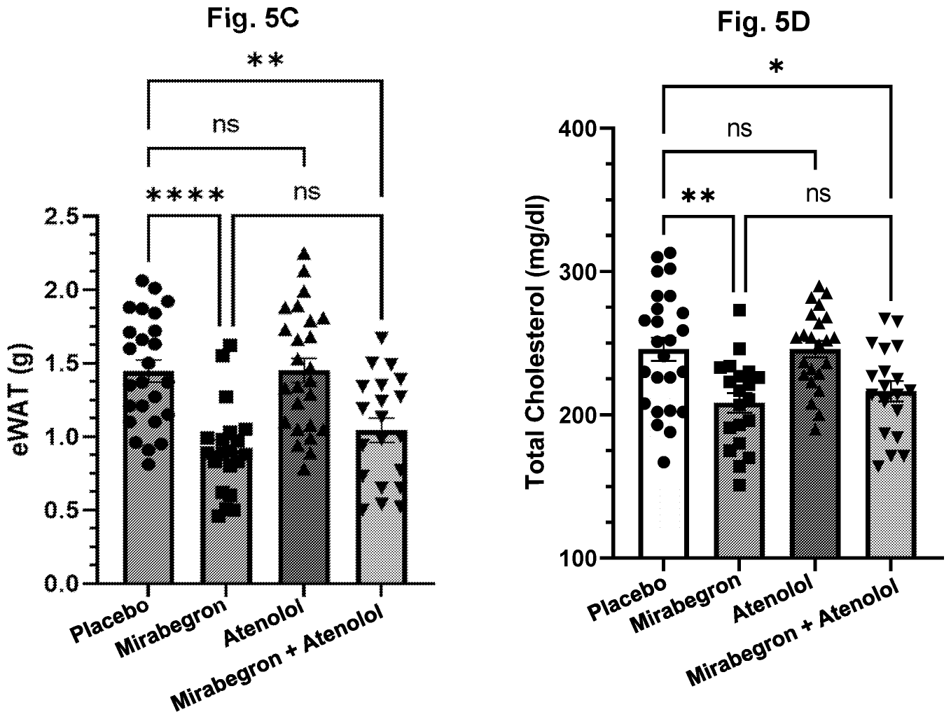

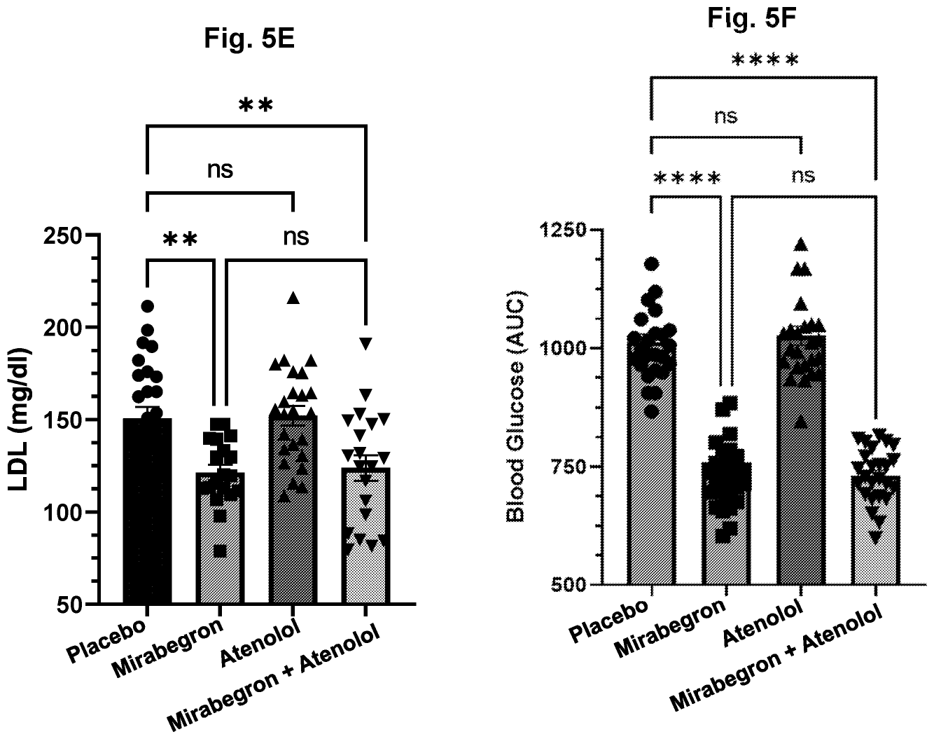

Mice treated with mirabegron alone (Group 2) and mirabegron co-administered with atenolol (Group 4) showed significant decreases in total body weight (FIG. 5A), total fat mass (FIG. 5B), epididymal fat mass (FIG. 5C), total cholesterol (FIG. 5D), and LDL cholesterol (FIG. 5E) compared to placebo (Group 1) and significant improvements in glucose tolerance, as indicated by reduced blood glucose area under the curve determined during formal glucose tolerance testing (FIG. 5F).

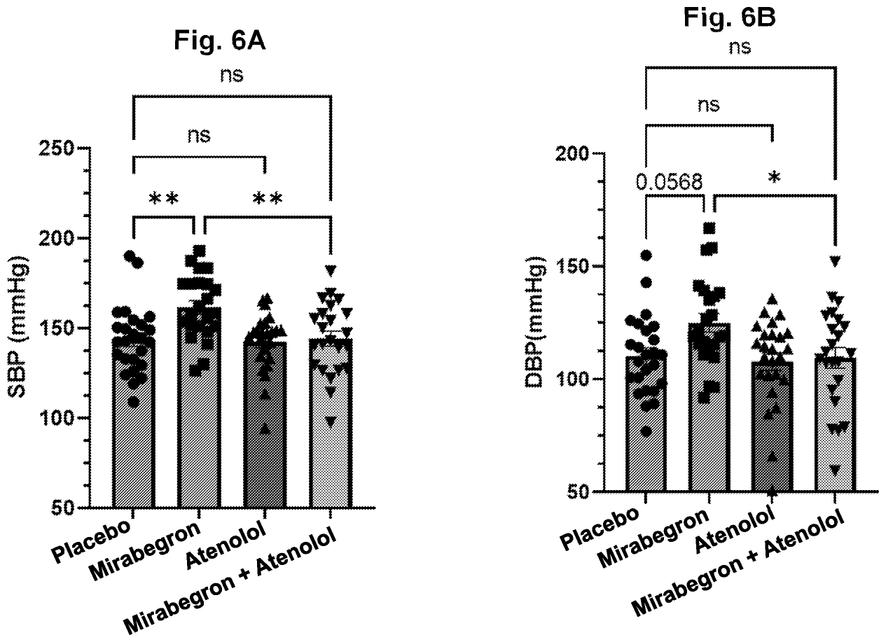

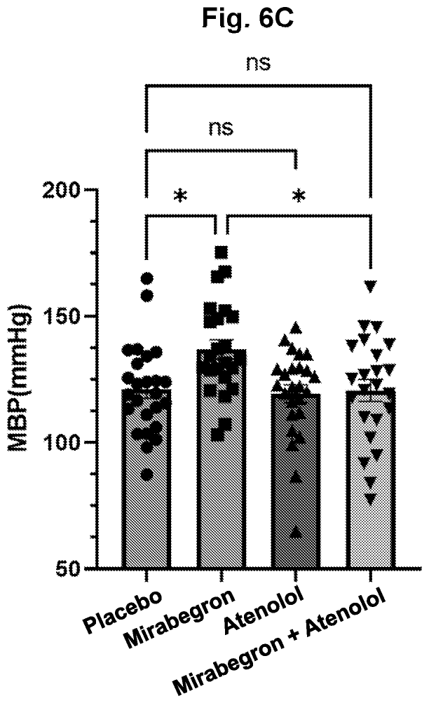

Mice treated with mirabegron alone (Group 2) had elevated systolic blood pressure (SBP), diastolic blood pressure (DBP), and mean arterial blood pressure (MBP) compared to placebo (FIGS. 6A-6C). Unexpectedly, mice treated with mirabegron and atenolol (Group 4) had blood pressure readings (SBP, DBP, and MBP) that were unchanged compared to placebo (FIGS. 6A-6C).

Together, data provided in Example 4 showed that a high dose of mirabegron (an ADRB3 agonist) had positive effects on mice by significantly reducing body weight, adiposity, total cholesterol, and LDL cholesterol and by significantly improving glucose tolerance. However, the high dose of mirabegron alone increased blood pressure in the mice-reflecting the known limitation of using a high dose of ADRB3 agonists to treat metabolic disorders in the clinic. Data also surprisingly showed that co-administration of a high dose of mirabegron and atenolol (an ADRB1 antagonist) did not change the beneficial effects of high-dose mirabegron treatment alone on body weight, adiposity, glucose tolerance, total cholesterol and LDL cholesterol but did preserve blood pressure at normal levels, comparable to placebo-treated mice. These data showed that administration of an ADRB1 antagonist combined with an ADRB3 agonist is not only effective for the treatment of metabolic disorders, but that by adding a ADRB1 antagonist to the treatment regimen, the amount of ADRB3 agonist that can be administered does not need to be lowered to protect the subject from untoward effects on the cardiovascular system.

1. A composition for treatment of a metabolic disorder in a subject in need thereof, the composition comprising at least one beta-1 adrenergic receptor (ADRB1) antagonist and at least one beta-3 adrenergic receptor (ADRB3) agonist.

2. The composition of claim 1, wherein the at least one ADRB1 antagonist comprises acebutolol, atenolol, betaxolol, bevantolol, bisoprolol, carvedilol, celiprolol, esmolol, labetalol, metoprolol, nadolol, nebivolol, penbutolol, pindolol, propranolol, sotalol, timolol, vortioxetine, or any combination thereof.

3. The composition of claim 1, wherein the at least one ADRB3 agonist comprises amibegron, BRL-37344, CL-316243, L-742791, L-796568, LY-368842, mirabegron, Ro40-2148, solabegron, vibegron, ritobegron, KUC-7322, or any combination thereof.

4. A pharmaceutical composition comprising the composition of claim 1, and at least one pharmaceutical acceptable excipient.

5. (canceled)

6. (canceled)

7. A method of treating and/or preventing a metabolic disorder in a subject in need thereof, the method comprising administering an effective amount of at least one beta-1 adrenergic receptor (ADRB1) antagonist and at least one beta-3 adrenergic receptor (ADRB3) agonist, wherein the subject has or is suspected of having a metabolic disorder.

8. The method of claim 7, wherein the metabolic disorder comprises type 1 diabetes mellitus, type 2 diabetes mellitus, impaired glucose tolerance (IGT), impaired fasting blood glucose (IFG), prediabetes, hyperglycemia, postprandial hyperglycemia, postabsorptive hyperglycemia, overweight, obesity, dyslipidemia, hyperlipidemia, hypercholesterolemia, hypertension, atherosclerosis, endothelial dysfunction, osteoporosis, chronic systemic inflammation, non-alcoholic fatty liver disease (NAFLD), retinopathy, neuropathy, nephropathy, polycystic ovarian syndrome, metabolic syndrome, or any combination thereof.

9. The method of claim 7, wherein administering an effective amount of the composition to a subject in need thereof, reduces blood glucose levels in the subject as compared to baseline, or increases plasma insulin levels in the subject as compared to baseline, or both.

10. (canceled)

11. A method for reducing the dosage of a beta-3 adrenergic receptor (ADRB3) agonist in a subject in need thereof, the method comprising administering to the subject an effective amount of one or more beta-1 adrenergic receptor (ADRB1) antagonists and one or more ADRB3 agonists, wherein the effective amount of the one or more ADRB3 agonists is less than the effective amount of the one or more ADRB3 agonist in a subject that is not receiving the one or more ADRB1 antagonists.

12. The method of claim 7, wherein the ADRB1 antagonist comprises acebutolol, acebutolol, atenolol, betaxolol, bevantolol, bisoprolol, carvedilol, celiprolol, esmolol, labetalol, metoprolol, nadolol, nebivolol, penbutolol, pindolol, propranolol, sotalol, timolol, vortioxetine, or any combination thereof.

13. The method of claim 7, wherein the ADRB3 agonist comprises amibegron, BRL-37344, CL-316243, L-742791, L-796568, LY-368842, mirabegron, Ro40-2148, solabegron, vibegron, ritobegron, KUC-7322, or any combination thereof.

14. A method of claim 11, wherein the effective amount of the one or more ADRB3 agonists causes an at least one untoward cardiovascular effect comprising undesired rise in blood pressure, heart rate, or both in a subject that is not receiving the one or more ADRB1 antagonists.

15. The method of claim 11, wherein the ADRB1 antagonist comprises acebutolol, acebutolol, atenolol, betaxolol, bevantolol, bisoprolol, carvedilol, celiprolol, esmolol, labetalol, metoprolol, nadolol, nebivolol, penbutolol, pindolol, propranolol, sotalol, timolol, vortioxetine, or any combination thereof and wherein the ADRB3 agonist comprises amibegron, BRL-37344, CL-316243, L-742791, L-796568, LY-368842, mirabegron, Ro40-2148, solabegron, vibegron, ritobegron, KUC-7322, or any combination thereof.

16.-17. (canceled)

18. The method of claim 11, wherein the administering to the subject an effective amount of one or more ADRB1 antagonists and one or more ADRB3 agonists occurs simultaneously.

19. The method of claim 11, wherein the administering to the subject an effective amount of one or more ADRB1 antagonists occurs immediately before the administering to the subject an effective amount of one or more ADRB3 agonists.

20. The method of claim 11, wherein the administering to the subject an effective amount of one or more ADRB1 antagonists occurs immediately after the administering to the subject an effective amount of one or more ADRB3 agonists.

21. The method of claim 14, wherein the least one untoward effect on the cardiovascular system resulting from ADRB3 agonist administration in a subject in need thereof comprises a mean arterial blood pressure (MBP) above normal, wherein a normal MBP ranges from 70 mmHg to 100 mmHg.

22. The method of claim 14, wherein the least one untoward effect on the cardiovascular system resulting from ADRB3 agonist administration in a subject in need thereof comprises a systolic arterial blood pressure (SBP) above normal, wherein normal SBP ranges from 120 mmHg to 140 mmHg.

23. The method of claim 14, wherein the least one untoward effect on the cardiovascular system resulting from ADRB3 agonist administration in a subject in need thereof comprises a diastolic arterial blood pressure (DBP) above normal, wherein normal DBP ranges from 70 mm Hg to 80 mmHg.

24. The method of claim 14, wherein the subject in need thereof has or is suspected of having a metabolic disorder.

25. (canceled)

26. The method of claim 7, wherein the method comprising administering to the subject an effective amount of one or more ADRB1 antagonists and one or more ADRB3 agonists reduces body fat in the subject, or reduces total cholesterol in the subject as compared to baseline, or reduces LDL cholesterol in the subject as compared to baseline or reduces triglycerides in the subject as compared to baseline or any combination thereof.

27-37. (canceled)
